(19) United States
(12) Patent Application Publication
(10) Pub. No.: US 2012/0088257 A1
(43) Pub. Date: Apr. 12, 2012

(54) METHOD FOR DIAGNOSING VASCULITIS

(75) Inventors: Luc Mouthon, Saint Mande (FR); Hanadi Dib, Fresnes (FR); Alexis Regent, Paris (FR)

(73) Assignee: Assistance Publique - Hopitaux de Paris, Paris (FR)

(21) Appl. No.: 13/203,098

(22) PCT Filed: Feb. 25, 2010
(86) PCT No.: PCT/FR2010/050331

§ 371 Date: Dec. 9, 2011

(30) Foreign Application Priority Data
Feb. 25, 2009 (FR) 0951205

(51) Int. Cl. G01N 33/566

(52) U.S. Cl. 435/7.92; 436/501

(57) ABSTRACT

The invention relates to a method for the in vitro detection of vasculitis or the risk of developing vasculitis, including determining the presence and/or the amount of anti-endothelial cell antibodies (AECA) or anti-vascular smooth muscle cell (VSMC) antibodies in a biological sample from a patient.

The invention relates to an in vitro method for detecting vasculitis or the risk of developing vasculitis, which comprises determining the presence and/or the amount of anti-endothelial cell antibodies (AECAs) or anti-vascular smooth muscle cell (VSMC) antibodies (Abs) in a biological sample from a patient.

### PRIOR ART

Vasculitis is an orphan pathological condition of which the prevalence is low, about 24 to 150 per million inhabitants. It represents a group of diseases characterized by the presence of inflammatory lesions on the vessel walls. Vasculitis is classified according to the size of the vessels affected and corresponds to various dominant pathogenic mechanisms: a production of pro-inflammatory cytokines and activation of macrophages in vasculitis involving the large-caliber vessels, such as Horton's diseases (or giant-cell arteritis); the deposition of circulating immune complexes responsible for activation of the conventional complement pathway, and the recruitment of neutrophils in vasculitis involving the medium-sized vessels, such as perarteritis nodosa associated with hepatitis B virus infection; and an activation of neutrophils by anti-neutrophil cytoplasmic antibodies (ANCAs), preferentially in vasculitis involving the small-caliber vessels, such as Wegener's granulomatosis and microscopic polyangiitis.

Horton's disease manifests itself through headaches due to the damage to the temporal artery, associated with a detrimental change in the general condition, with rhizomelic pseudopolyarthritis in one out of two cases and, in the vast majority of cases, with an inflammatory syndrome.

Wegener's granulomatosis is a granulomatosis vasculitis involving especially the sinuses of the face, the lungs and the kidneys, associated in the systemic forms, in 90% of cases, with anti-proteinase 3 Abs.

Microscopic polyangiitis is a necrotizing vasculitis which involves the small-caliber vessels and may be responsible for glomerula and lung capillary damage responsible for a pneumorenal syndrome, in addition to the systematic manifestations in connection with vasculitis.

Churg-Strauss syndrome corresponds to a late-onset asthma with severe progression, associated with hypereosinophilia and necrotizing vasculitis.

Currently, the diagnosis of ANCA-positive vasculitis is still based on a biopsy, whether it is a skin biopsy, a renal biopsy, a neuromuscular biopsy, or the like. ANCAs constitute an important aid to the diagnosis of systemic vasculitis. Anti-myeloperoxidase (MPO) ANCAs are thus present in 60 to 75% of patients suffering from microscopic polyangiitis and 38% of patients suffering from Churg-Strauss syndrome. Consequently, a notable proportion of patients do not have ANCAs, which makes diagnosis, evaluation of prognosis and therapeutic treatment difficult.

At the current time, there is no identified biological marker over the course of Horton's disease. The diagnosis of Horton's disease is based, in the majority of cases, on a temporal artery biopsy, which is positive in 80% of cases of Horton's disease.

There thus exists a need to identify immunological markers that are of diagnostic and/or prognostic interest over the course of Horton's disease and immunological markers, other than ANCAs, that are of interest over the course of ANCA-positive vasculitis. International application WO 2004/094638, in Japanese, mentions a search for anti-peroxiredoxin 2 antibodies in the serum of patients suffering from vasculitis, but appears to remain the only one in this field.

In this perspective, the inventors have focused on anti-endothelial cell antibodies (AECAs) and anti-vascular smooth muscle cell (VSMC) antibodies (Abs). In particular, AECAs are detected and appear to play a key role in the pathogenesis of vasculitis (Guilpain and Mouthon, Clinic Rev Allerg Immunol, 2008 October; 35(1-2):59-65). Thus, the binding of AECAs to endothelial cells can lead to destruction of the target cell via an Ab-dependent cell cytotoxicity (ADCC) mechanism, and can induce apoptosis and increase the expression of adhesion molecules. However, the antigenic targets of these antibodies have not up until now been identified.

### SUMMARY OF THE INVENTION

The inventors have now identified antigenic targets of anti-endothelial cell antibodies (AECAs) and of anti-vascular smooth muscle cell (VSMC) antibodies in vasculitis, in particular in Horton's disease and ANCA-positive vasculitis.

On this basis, the invention provides an in vitro method for detecting vasculitis in an individual, or the risk of developing vasculitis, which comprises determining the presence and/or the amount of at least one AECA or of an anti-VSMC Ab, directed against an antigen chosen from the group consisting of vinculin, FUbp2 (far upstream element-binding protein 2), caldesmon, 78 kDa glucose-regulating protein precursor, heat shock cognate 71 kDa protein, stress protein 70 mitochondrial precursor, lamin-A/C, heterogeneous nuclear ribonucleoprotein K, T-complex protein 1 subunit epsilon, 60 kDa heat shock protein mitochondrial precursor, protein disulfide isomerase A1 precursor, protein disulfide isomerase A3 precursor, T-complex protein 1 subunit theta, T-complex protein 1 subunit beta, ATP synthase subunit alpha mitochondrial precursor, heterogeneous nuclear ribonucleoprotein H, tubulin beta-chain, fructose-bisphosphate aldolase A, ATP synthase subunit alpha mitochondrial precursor, calumenin precursor, reticulocalbin-3, 26S proteasome non-ATPase regulatory subunit 13, inorganic pyrophosphatase, annexin A5, 14-3-3 protein epsilon, 6-phosphogluconolactonase, galectin-1, succinyl-CoA:3-keto acid-coenzyme A transferase 1 mitochondrial precursor, heterogeneous nuclear ribonucleoprotein D0, 26S protease regulatory subunit 7, heme oxygenase 2, histon H2B type F-S, proteasome subunit alpha type-5, proteasome subunit beta type-2, cytoskeleton-associated protein 4, uroporphyrinogen decarboxylase, adenine phosphoribosyltransferase, profilin-1, plastin-3, growth factor receptor-bound protein 2, heterogeneous nuclear ribonucleoprotein L, reticulocalbin-1 precursor, calumenin precursor, serpinB9, isocitrate dehydrogenase [NAD] subunit alpha mitochondrial precursor, GMP synthase [glutamine-hydrolyzing], T-complex protein 1 subunit zeta, cofilin-1, aconitate hydratase mitochondrial precursor, mitochondrial inner membrane protein, heterogeneous nuclear ribonucleoprotein K, elongation factor Tu mitochondrial precursor, alcohol dehydrogenase [NADP+], sialic acid synthase, S-formylglutathione hydrolase, guanine nucleotide-binding protein subunit beta-2-like 1, purine nucleoside phosphorylase, prohibitin, C1q-binding protein mitochondrial precursor, transitional endoplasmic reticulum ATPase, nucleoside diphosphate kinase A, alpha-enolase, nucleophosmin, annexin A2, ADP-ribosylation factor-like protein 6-interacting protein 4, FUbp1 (far upstream element-binding protein 1), dihydrolipoyl dehydrogenase mitochondrial precursor, inosine-5′-monophosphate dehydrogenase 2, tripeptidyl-peptidase 1 precursor, fumarate hydratase mitochondrial precursor, heterogeneous nuclear ribonucleoprotein D0, PDZ and LIM domain protein 1, 60S acidic ribosomal protein P0, voltage-dependent anion-selective channel protein 2, DJ-1 protein, peptidyl-prolyl cis-trans isomerase A, thioredoxin-dependent peroxide reductase mitochondrial precursor, T-complex protein 1 subunit beta, DNAJ homolog subfamily B member 11 precursor, glutaredoxin-3, Rho GDP dissociation inhibitor protein 2, and glutathione S-transferase P, in a biological sample from a patient, the presence of said at least one antibody being an indicator of vasculitis or of the risk of developing vasculitis.

Said vasculitis may in particular be Wegener's granulomatosis, microscopic polyangiitis or Churg-Strauss syndrome.

Said vasculitis may also be Horton's disease.

Preferably, the presence of said at least one antibody in the biological sample is compared with a control value, the presence of said at least one antibody in an amount greater than the control value being an indicator of vasculitis or of the risk of developing vasculitis.

Another subject of the invention is an in vitro method for the prognosis or monitoring of vasculitis, which comprises determining the presence and/or the amount of at least one antibody as defined above, in a biological sample from a patient, at various times, an increase in the amount of said at least one antibody over time being an indicator of a worsening of the vasculitis.

Another subject of the invention is an in vitro method for evaluating the efficacy of a treatment for vasculitis, which comprises determining the presence and/or the amount of at least one antibody as defined above, in a biological sample from a patient, at various times before, during or after the treatment, a decrease in the amount of said at least one antibody over time being an indicator of an improvement of the vasculitis.

### DETAILED DESCRIPTION OF THE INVENTION

The inventors have used normal human umbilical vein endothelial cells (HUVECs) as a source of antigens and tested the sera of patients having Horton's disease, or systemic vasculitis associated with anti-neutrophil cytoplasmic antibodies (ANCAs), and healthy individuals.

In order to identify the targets of the antibodies, the inventors have used two-dimensional immunoblocking, the antigens being identified by mass spectrometry.

The inventors have also tested the sera of patients suffering from Horton's disease.

### DEFINITIONS

The term “biological sample” refers to any biological sample from a patient. Examples of samples include biological fluids and tissue biopsies. Preferentially, the sample may be blood, serum, saliva, urine or sperm. More preferably, the biological sample is a blood or serum sample.

The term “patient” refers to any individual capable of being tested. Preferably, it is a human being, but the term includes any other mammal, such as dogs, cats, rodents, cattle, horses, monkeys, etc. The patient can be tested regardless of his or her sex or age. The patient may be an individual at risk, may be asymptomatic or may exhibit early or advanced signs of vasculitis.

The term “diagnosis” means the identification of the pathological condition or the evaluation of the state of severity of the pathological condition.

The term “prognosis” means the evaluation of the risk of worsening, and of the consequences thereof.

The term “control value” refers to a basal value corresponding to the mean of the values obtained with the biological sample from healthy individuals, not affected by vasculitis or a disease capable of causing vasculitis. It may be a reference statistical value.

In order to evaluate the progression of the pathological condition, it may be useful to test a patient and to verify the effect of a treatment or the progression of the pathological condition by testing the patient again, for example after a gap of several months. In this case, the results of the second test are compared with the results of the first test, and also often with the “control” value.

An amount of antibodies “greater than the control value” generally means a statistically significant increase, for example of at least two standard deviations above the mean of the optical densities of the IgG reactivities of all the healthy individuals.

The term “capture antigen” is intended to mean an antigen, preferably attached to a solid phase, which is capable of retaining said at least one antibody present in a biological sample, by affinity binding. The capture antigen may be labeled.

The term “label” refers both to direct labeling (by means of enzymes, radioisotopes, fluorochromes, luminescent compounds, etc.) and to indirect labeling (for example by antibodies which are themselves labeled directly, or by means of reagents of a labeled “affinity pair”, such as, but not exclusively, the label avidin-biotin pair, etc.

The term “vasculitis” is intended to mean any primary systemic vasculitis and also secondary vasculitis, in particular drug-related vasculitis, vasculitis associated with a connective tissue disorder, or vasculitis of infectious origin. The vasculitis targeted thus includes vasculitis affecting the small-caliber vessels, such as Wegener's granulomatosis, microscopic polyangiitis and Churg-Strauss syndrome, vasculitis affecting the medium-caliber vessels, such as periarteritis nodosa, and vasculitis affecting the large-caliber vessels, such as Horton's disease.

### Antibodies Identified:

As indicated in the “examples” section, the inventors have identified several anti-endothelial cell antibodies (AECAs) or anti-vascular smooth muscle cell (VSMC) antibodies in patients with vasculitis.

These antigenic targets are involved in particular in oxidative stress, cell metabolism and the maintenance of cell homeostasis.

The detection and/or quantification of these antibodies can be carried out for detecting vasculitis, for giving a prognosis for or monitoring these pathological conditions, or for evaluating the efficacy of a treatment for these pathological conditions.

The antigens recognized by the antibodies recognized are listed below (cf. also tables 1 to 9, of the “Examples” section). A listing of these protein sequences is also appended.

The accession numbers in the SwissProt database and the corresponding sequences are given by way of indication.

### Vinculin (Swiss-Prot: P18206, SEQ ID NO:1)

FUbp2 (far upstream element-binding protein 2) (Swiss-Prot: Q92945, SEQ ID NO:2)

### Caldesmon (Swiss-Prot: Q92945, SEQ ID NO:3)

78 kDa glucose-regulated protein precursor (Swiss-Prot: P11021, SEQ ID NO:4)
Heat shock cognate 71 kDa protein (Swiss-Prot: P11142, SEQ ID NO:5)
Stress protein 70 mitochondrial precursor (Swiss-Prot: P38646, SEQ ID NO:6)

### Lamin-A/C (Swiss-Prot: P02545, SEQ ID NO:7)

Heterogeneous nuclear ribonucleoprotein K (Swiss-Prot: P61978, SEQ ID NO:8)
T-complex protein 1 subunit epsilon (Swiss-Prot: P48643, SEQ ID NO:9)
60 kDa heat shock protein mitochondrial precursor (Swiss-Prot: P10809, SEQ ID NO:10)
Protein disulfide isomerase A1 precursor (Swiss-Prot: P07237, SEQ ID NO:11)
Protein disulfide isomerase A3 precursor (Swiss-Prot: P30101, SEQ ID NO:12)
T-complex protein 1 subunit theta (Swiss-Prot: 50990, SEQ ID NO:13)
T-complex protein 1 subunit beta (Swiss-Prot: P78371, SEQ ID NO:14)
ATP synthase subunit alpha mitochondrial precursor (Swiss-Prot: P25705, SEQ ID NO:15)
Heterogeneous nuclear ribonucleoprotein H (Swiss-Prot: P31943, SEQ ID NO:16)
Tubulin beta-chain (Swiss-Prot: P07437, SEQ ID NO:17)
Fructose-bisphosphate aldolase A (Swiss-Prot: P04075, SEQ ID NO:18)
Calumenin precursor (Swiss-Prot: O43852, SEQ ID NO:19)

### Reticulocalbin-3 (Swiss-Prot: Q96D15, SEQ ID NO:20)

26S proteasome non-ATPase regulatory subunit 13 (Swiss-Prot: Q9UNM6, SEQ ID NO:21)
Inorganic pyrophosphatase (Swiss-Prot: Q15181, SEQ ID NO:22)

### Annexin A5 (Swiss-Prot: P08758, SEQ ID NO:23)

14-3-3 protein epsilon (Swiss-Prot: P62258, SEQ ID NO:24)
6-phosphogluconolactonase (Swiss-Prot: O95336, SEQ ID NO:25)

### Galectin-1 (Swiss-Prot: P09382, SEQ ID NO:26)

Succinyl-CoA:3-keto acid-coenzyme A transferase 1 mitochondrial precursor (Swiss-Prot: P55809, SEQ ID NO:27)
Heterogeneous nuclear ribonucleoprotein D0(Swiss-Prot: Q14103, SEQ ID NO:28)
26S protease regulatory subunit 7 (Swiss-Prot: P35998, SEQ ID NO:29)
Heme oxygenase 2 (Swiss-Prot: P30519, SEQ ID NO:30)
Histone H2B type F-S (Swiss-Prot: P57053, SEQ ID NO:31)
Proteasome subunit alpha type-5 (Swiss-Prot: P28066, SEQ ID NO:32)
Proteasome subunit beta type-2 (Swiss-Prot: P49721, SEQ ID NO:33)
Cytoskeleton-associated protein 4 (Swiss-Prot: Q07065, SEQ ID NO:34)
Uroporphyrinogen decarboxylase (Swiss-Prot: P06132, SEQ ID NO:35)
Adenine phosphoribosyltransferase (Swiss-Prot: P07741, SEQ ID NO:36)

### Profilin-1 (Swiss-Prot: P07737, SEQ ID NO:37)

### Plastin-3 (Swiss-Prot: P13797, SEQ ID NO:38)

Growth factor receptor-bound protein 2 (Swiss-Prot: P62993, SEQ ID NO:39)
Heterogeneous nuclear ribonucleoprotein L (Swiss-Prot: P14866, SEQ ID NO:40)
Reticulocalbin-1 precursor (Swiss-Prot: Q15293, SEQ ID NO:41)

### Serpin B9 (Swiss-Prot: P50453, SEQ ID NO:42)

Isocitrate dehydrogenase [NAD] subunit alpha mitochondrial precursor (Swiss-Prot: P50213, SEQ ID NO:43)
GMP synthase [glutamine-hydrolyzing] (Swiss-Prot: P49915, SEQ ID NO:44)
T-complex protein 1 subunit zeta (Swiss-Prot: P40227, SEQ ID NO:45)

### Cofilin-1 (Swiss-Prot: P23528, SEQ ID NO:46)

Aconitate hydratase mitochondrial precursor (Swiss-Prot: Q99798, SEQ ID NO:47)
Mitochondrial inner membrane protein (Swiss-Prot: Q16891, SEQ ID NO:48)
Heterogeneous nuclear ribonucleoprotein K (Swiss-Prot: P61978, SEQ ID NO:49)
Elongation factor Tu mitochondrial precursor (Swiss-Prot: P49411, SEQ ID NO:50)
Alcohol dehydrogenase [NADP+] (Swiss-Prot: P14550, SEQ ID NO:51)
Sialic acid synthase (Swiss-Prot: Q9NR45, SEQ ID NO:52)
S-formylglutathione hydrolase (Swiss-Prot: P10768, SEQ ID NO:53)
Guanine nucleotide-binding subunit beta-2-like 1 (Swiss-Prot: P63244, SEQ ID NO:54)
Purine nucleoside phosphorylase (Swiss-Prot: P00491, SEQ ID NO:55)

### Prohibitin (Swiss-Prot: P35232, SEQ ID NO:56)

C1q-binding protein mitochondrial precursor (Swiss-Prot: Q07021, SEQ ID NO:57)
Transitional endoplasmic reticulum ATPase (Swiss-Prot: P55072, SEQ ID NO:58)
and Nucleoside diphosphate kinase A (Swiss-Prot: P15531, SEQ ID NO:59)
and also

### Annexin A2 (Swiss-Prot: P07355, SEQ ID NO:60)

### Alpha-enolase (Swiss-Prot: P06733, SEQ ID NO:61)

FUbp1 (far upstream element-binding protein 1) (Swiss-Prot: Q96AE4, SEQ ID NO:62)
Dihydrolipoyl dehydrogenase mitochondrial precursor (Swiss-Prot: P09622, SEQ ID NO:63)
Inosine-5′-monophosphate dehydrogenase 2 (Swiss-Prot: P12268, SEQ ID NO:64)
Tripeptidyl-peptidase 1 precursor (Swiss-Prot: P014773, SEQ ID NO:88)
Fumarate hydratase mitochondrial precursor (Swiss-Prot: P07954, SEQ ID NO:65)
Heterogeneous nuclear ribonucleoprotein D0(Swiss-Prot: Q14103, SEQ ID NO:66)
PDZ and LIM domain protein 1 (Swiss-Prot: O00151, SEQ ID NO:67)
60S acidic ribosomal protein P0 (Swiss-Prot: P05388, SEQ ID NO:68)
Voltage-dependent anion-selective channel protein 2 (Swiss-Prot: P45880, SEQ ID NO:69)
DJ-1 protein (Swiss-Prot: Q99497, SEQ ID NO:70) Peptidyl-prolyl cis-trans isomerase A (Swiss-Prot: P62937, SEQ ID NO:71)
Thioredoxin-dependent peroxide reductase mitochondrial precursor (Swiss-Prot: P30048, SEQ ID NO:72)
DNAJ homolog subfamily B member 11 precursor (Swiss-Prot: Q9UBS4, SEQ ID NO:73)

### Glutaredoxin-3 (Swiss-Prot: P076003, SEQ ID NO:74)

Rho GDP dissociation inhibitor protein 2 (Swiss-Prot: P52566, SEQ ID NO:75)

### Glutathione S-transferase P (Swiss-Prot: P09211, SEQ ID NO:76)

### and Peroxyredoxin (Swiss-Prot: P32119, SEQ ID NO:77)

and also
Putative heat shock protein HSP 90-alpha A2 (Swiss-Prot: Q14568, SEQ ID NO:78)
Coatomer subunit alpha (Swiss-Prot: P53621, SEQ ID NO:79)
UDP-glucose 6-dehydrogenase (Swiss-Prot: O60701, SEQ ID NO:80)
Actin, cytoplasmic 1 (Swiss-Prot: P60709, SEQ ID NO:81)
POTE akyrin domain family member E (Swiss-Prot: Q6S8J3, SEQ ID NO:82)

### Nucleophosmin (Swiss-Prot: P06748, SEQ ID NO:83)

Elongation factor 2 (Swiss-Prot: P13639, SEQ ID NO:84)
DnaJ homolog subfamily A member 1 (Swiss-Prot: P31689, SEQ ID NO:85)
Actin, cytoplasmic 2 (Swiss-Prot: P63261, SEQ ID NO:86)
26S protease regulatory subunit 8 (Swiss-Prot: P62195, SEQ ID NO:87).

Among the autoantibodies detected, several are particularly relevant. They are the antibodies directed against the following antigens:

### Caldesmon

78 kDa glucose-regulated protein precursor
Heat shock cognate 71 kDa protein
T-complex protein 1 subunit epsilon
Protein disulfide isomerase A3 precursor
or
Calumenin precursor,
and especially the antibodies directed against vinculin
or lamin.

The first six antigens are recognized by more than 60% of the pools of the three sera of patients suffering from Wegener's granulomatosis that were tested, two of them (caldesmon and calumenin precursor) also being recognized by the pools of three sera of patients having Churg-Strauss syndrome without ANCAs.

The antibodies identified by the inventors can be used in the methods according to the invention alone or in combination. The detection and/or the quantification can be carried out with respect to just one of the antibodies identified, or can relate to a plurality of antibodies. It is thus possible to imagine the method being carried out on a solid support, for example a microplate, on which the antigens corresponding to the plurality of antibodies to be detected and/or quantified are arranged in a defined and ordered manner.

According to one embodiment of the invention, the methods described implement the detection of an antibody directed against an antigen identified in table 1, 8 or 9, for the diagnosis, prognosis or monitoring of a Wegener's granulomatosis.

More particularly, the invention relates to a method for the diagnosis, prognosis or monitoring of a Wegener's granulomatosis, which method comprises detecting an antibody directed against an antigen chosen from caldesmon, 78 kDa glucose-regulated protein precursor, heat shock cognate 71 kDa protein, T-complex protein 1 subunit epsilon, protein disulfide isomerase A3 precursor, or calumenin precursor.

According to another embodiment of the invention, the methods described implement the detection of an antibody directed against an antigen identified in table 2 of table 5, for the diagnosis, prognosis or monitoring of a microscopic polyangiitis. More particularly, the methods described may use the detection of an antibody directed against an antigen identified in table 2, 8 or 9, for the diagnosis, prognosis or monitoring of a microscopic polyangiitis with anti-MPO ANCAs. Moreover, the methods described may use the detection of an antibody directed against an antigen identified in table 5, 8 or 9, for the diagnosis, prognosis or monitoring of a microscopic polyangiitis without anti-MPO ANCAs.

According to another embodiment of the invention, the methods described implement the detection of an antibody directed against an antigen identified in table 3 or 4, 8 or 9, for the diagnosis, prognosis or monitoring of a Churg-Strauss syndrome.

More particularly, the methods described implement the detection of an antibody directed against an antigen identified in table 3, 8 or 9, for the diagnosis, prognosis or monitoring of a Churg-Strauss syndrome with anti-MPO ANCAs.

Moreover, the methods described may use the detection of an antibody directed against an antigen identified in table 4, for the diagnosis, prognosis or monitoring of a Churg-Strauss syndrome without anti-MPO ANCAs.

According to another embodiment of the invention, the methods described implement the detection of an antibody directed against an antigen identified in one of tables 6, 7, 8 or 9, for the diagnosis, prognosis or monitoring of Horton's disease, the antigen preferably being vinculin or lamin.

### Assaying of Antibodies:

The biological sample is preferably a serum sample, preferably diluted to 1/100th, or more, for example to 1/200th or 1/400th.

Advantageously, the amount of antibodies can be determined by means of an immunoassay.

The biological sample may be optionally treated in a prior step, or brought directly into contact with at least one capture antigen.

The method according to the invention may be carried out according to various formats well known to those skilled in the art: in solid phase or in homogeneous phase; in one step or in two steps; in a competitive method, by way of nonlimiting examples.

According to one preferred embodiment, the capture antigen is immobilized on a solid phase. By way of nonlimiting examples of a solid phase, use may be made of microplates, in particular polystyrene microplates, such as those sold by the company Nunc, Denmark. Use may also be made of solid particles or beads, paramagnetic beads, such as those supplied by Dynal or Merck-Eurolab (France) (under the trademark Estapor™), or else test tubes made of polystyrene or polypropylene, etc.

An immunoassay format for detecting the antibodies by competition is also possible. Other immunoassay modes can also be envisioned and are well known to those skilled in the art.

ELISA assays, radioimmunoassays, or any other detection technique can be used to reveal the presence of the antigen-antibody complexes formed.

According to one particular preferred embodiment, the capture antigen corresponds to a whole protein or to a fragment of said protein. For example, the method of the invention comprises bringing a biological sample into contact with a whole protein recognized by the antibody to be detected and/or quantified.

In one particular example, the capture antigen may be coupled to a glutathione S-transferase (GST), before being deposited on a microplate.

By way of illustration, the serum samples to be tested, for example diluted to 1/100th, are incubated on the microplate. After washing, labeled anti-human Fcγ antibodies (for example labeled with an alkaline phosphatase) are added, the complexes being revealed (for example by adding a substrate for the phosphatase, the cleavage of which can be detected by reading the absorbance).

### Patients Targeted:

The patients targeted are suffering from vasculitis, are suspected of suffering from vasculitis or are liable to develop vasculitis.

This may involve vasculitis in ANCA-positive patients, or in patients who do not have ANCA autoantibodies.

The methods of the invention make it possible to diagnose, give a prognosis for or monitor the progression of any type of vasculitis, in particular Wegener's granulomatosis, microscopic polyangiitis, Churg-Strauss syndrome, or Horton's disease.

### Evaluation of the Efficacy of a Treatment:

Another subject of the invention is an in vitro method for evaluating the efficacy of a treatment for vasculitis, which comprises determining the presence and/or the amount of at least one antibody as defined above in a biological sample from a patient, at various times before, during or after the treatment, a decrease in the amount of said at least one antibody over time being an indicator of an improvement of the vasculitis.

The following examples illustrate the invention without limiting the scope thereof.

### EXAMPLES

### Example 1

### Characterization of the Antigenic Targets of The AECAs in ANCA-Positive Vasculitis

The sera of 45 patients having ANCA-positive vasculitis (15 having Wegener's granulomatosis (WG), 12 having microscopic polyangiitis (MPA), 12 having Churg-Strauss syndrome (CSS)) were tested in pools of three and compared with a pool of sera of 12 healthy individuals. The serum IgG reactivities were analyzed using two-dimensional electrophoresis gels followed by immunoblotting using normal human umbilical vein endothelial cell (HUVEC) antigens (cf. Servettaz et al, Proteomics. 2008 March; 8(5):1000-8).

The serum IgGs of the pools of patients suffering from WG with anti-proteinase 3 (PR3) ANCAs (n=5), MPA with anti-myeloperoxidase (MPO) ANCAs (n=2), MPA without anti-MPO ANCAs (n=2), CSS with anti-MPO ANCAs (n=1) and CSS without anti-MPO ANCAs (n=2), recognized 107±17, 148, 211, 128 and 101 protein spots, respectively, whereas the serum IgGs of healthy individuals recognized 79 protein spots. The serum IgGs of patients suffering from WG with anti-PR3, MPA with anti-MPO, MPA without anti-MPO, CSS with anti-MPO and CSS without anti-MPO specifically recognized 37, 12, 22, 15 and 23 protein spots, respectively. The target antigens were involved in oxidative stress, cellular metabolism and other key biological cell functions.

### Method of Identification:

### Two-Dimensional Electrophoresis

The inventors used a pH of 3 to 10 and an acrylamide gradient of 7% to 18% in all the experiments, which made it possible to study a large amount of antigens from 10 to 200 kDa.

The proteins were subjected to isoelectric focusing on the Protean IEF Cell System, as described in Görg et al, 2000, Electrophoresis, 21(6):1037-53.

Briefly, immediately after the isoelectric focusing, the samples were thawed and diluted in IPG buffer containing 7M ultrapure urea (VWR, Fontenay-Sous-Bois, France), 2M thiourea (Sigma), 4% CHAPS (Sigma), 0.002% Triton X100 (Sigma), 60 μl of ampholyte vehicle pH 3-10 (Pharmalytes 3-10, Amersham Biosciences, Uppsala, Sweden) and bromophenol blue (Sigma). To prepare the 2-D gels, 100 μg of HUVEC proteins were loaded onto the IPG strips. The latter were rehydrated and subjected to automated electrophoresis for 12 h at 50 V, 1 h at 200 V, 1 h at 1000 V and 7 h at 10 000 V (6 h linear and 1 h rapid).

Before the second dimension, the strips were equilibrated for 15 min in 10 ml of the first equilibration solution (51 mM Tris [Amersham Biosciences], 6 mM urea, 40% (v/v) glycerol, 52 mM SDS [Amersham Biosciences], 32.4 mM DTT), and then for 20 min in a second equilibration solution (51 mM Tris, 6 mM urea, 40% [v/v] glycerol, 52 mM SDS, 86.5 mM iodoacetamide). The equilibrated strips were transferred onto the 7%-18% polyacrylamide gradient gel. Ten microliters of Precision Plus Protein Unstained Standards molecular weight (MW) markers (Bio-Rad) were loaded onto each gel. The second dimension was performed on a Laemmli system on 7%-18% linear gradient polyacrylamide gels (20 cm×20 cm×1.5 mm): a solution containing 18.5% of 2.5M PAGE acrylamide (Amersham Biosciences), 24.7 mM piperazine diacrylamide/diacrylyl (PDA) (Bio-Rad), 0.375M Tris-HCl (Amersham Biosciences) pH 8.8, 15% (v/v) glycerol (Sigma), 3.5 mM SDS, 0.05% (v/v) TEMED (Bio-Rad) and 1.6 mM ammonium persulfate (APS) (Bio-Rad), and a solution containing 7% of 1.0M acrylamide, 10 mM PDA, 0.375M Tris-HCl pH 8.8, 3.5 mM SDS, doubly-distilled water, 0.06% (v/v) TEMED and 2.4 mM APS were mixed. The equilibrated IPG gels were sealed over the polyacrylamide gels with 1% of agarose containing bromophenol blue, and electrophoresis buffer (24.8 mM Tris, 192 mM glycine, and 0.1% SDS) was added. The gels were subjected to an electrophoresis initially at 40 V (constant) for 1 h and then at 15 mA/gel for 21 h 15 min.

### Electrotransfer and Immunoblotting

The gels were transferred onto PVDF membranes (Millipore, Bedford, Mass., USA) by semi-dry transfer (Bio-Rad) at 320 mA for 1 h 30 min. After blocking with PBS-0.2% Tween for 90 min, the membranes were incubated overnight at 4° C. with the pools of sera of three phenotypically identical patients (Wegener's granulomatosis, microscopic polyangiitis or Churg-Strauss syndrome) and the pools of sera of 14 healthy blood donors, at a dilution of 1:100. The membranes were washed before incubation with a rabbit anti-human Fcγ second Ab coupled to alkaline phosphatase (Dako, Glostrup, Denmark) for 90 min at ambient temperature. The immunoreactivities were revealed using an NBT-BCIP substrate (Sigma). The specific reactivities were determined by densitometry (GS-800, Bio-Rad) using the Quantity one software (Bio-Rad). The membranes were then stained with colloidal gold (Protogold, British Biocell International, Cardiff, UK) and subjected to a second densitometric analysis in order to record the spots of labeled proteins for each gel.

### Gel Labeling

The analytical gels were stained with ammoniacal silver nitrate.

### Gel Image Analysis and 2-D Transfers

The images of the gels and of the membranes obtained using the GS-800 densitometer (Bio-Rad) were analyzed by means of the Image Master 2-D Platinum 6 system (Amersham Biosciences), before and after staining with colloidal gold. The specific labelings were manually linked up with the IgG-probed protein spots on the two images. The algorithm automatically transferred these labelings of the image of the 2-D blot stained with colloidal gold to the images of the gels stained with silver nitrate.

Digestion of the Gel with Trypsin

The digestion of the gel was carried out by the Freedom EVO 100 digester/spotter robot (Tecan, Männedorf, CH). The spots were destained twice with a mixture of 100 mM ammonium bicarbonate (ABC) and 50% ACN for 45 min at 22° C. and then dried with 100% ACN for 15 min. They were then subjected to treatment with 25 mM ABC containing 10 mM DTT for 1 h at 60° C. and then subsequently alkylated with 55 mM iodoacetamide in 25 mM ABC for 30 min in the dark at 22° C. The pieces of gel were washed twice in 25 mM ABC and reduced twice in 100% ACN for 15 min and dried in 100% ACN for 10 min. The strips were completely dehydrated after 1 h at 60° C. The pieces of gel were incubated in 13 μl of trypsin (Sequencing Grade Modified Trypsin from Promega, Wis., USA; 12.5 μg/ml in 40 mM ABC-10% ACN, pH 8.0) overnight at 40° C. After digestion, the peptides were washed with 30 μl of 25 mM ABC, reduced with 100% ACN and extracted twice with a mixture of 50% ACN-5% formic acid (FA). The extracts were subsequently dried by centrifugation under vacuum (Eppendorf, Hamburg, Germany). Finally, the peptides were desalted using C18-ZipTips (Millipore) and two elutions, the first with 50% ACN-5% FA, and then with 80% ACN-5% FA. The combined elutions were dried at ambient temperature.

### Protein Identification by Mass Spectrometry (MS)

For the MS and MS/MS analyses, the peptides were redissolved in 4 μl CHCA (5 mg/ml in 50% ACN-0.1% TFA). One and a half microliters of each sample were deposited directly on a MALDI plate (Applied Biosystems, Foster City, Calif., USA). The drops were dried at ambient temperature. The analysis of the samples used a MALDI-TOF-TOF 4800 mass spectrometer (Applied Biosystems). The acquisition of the spectra and their processing were carried out by means of the 4000 series explorer software (Applied Biosystems) version 3.5.28193. The external calibration of the plate was carried out by means of four points deposited at the four corners of the plate with a mixture of five external standards (PepMix 1, LaserBio Labs, Sophia Antipolis, France). The peptide masses were acquired in steps of 50 spectra of 900 to 4000 Da. The MS spectra were produced by addition using 1000 laser shots with an Nd-YAG laser operating at 355 nm and 200 Hz. After filtration of the contaminating trypsin, keratin and matrix peaks, up to 15 parent ions were selected for a subsequent MS/MS fragmentation, according to their mass, the intensity of the signal, the signal-to-noise ratio, and the absence of neighboring masses in the MS spectrum. The MS/MS spectra were acquired in 1 kV positive mode, and 1000 shots were added together 50 by 50. The search on databases was carried out by means of the Mascot 2.2 software (MatrixScience, London, UK) via GPS explorer (Applied Biosystems) version 3.6 combining the MS and MS/MS interrogations on the human proteins of the Swissprot 54.5 library (www.expasy.org). The search parameters were the following: possible carbamidomethylation of cysteines and possible oxidation of methionines. Up to one missed tryptic cleavage was permitted, and a tolerance of 30 ppm for the accuracy of the mass for the precursors, and 0.3 Da for the fragments was permitted for all the tryptic mass searches.

The identification was based on a Mascot score above the level of significance (i.e. <5%). In the case where peptides correspond to multiple members of a protein family, the protein reported is that with the greatest number of correspondences (peptide matches).

### Results:

The inventors identified 37 protein spots corresponding to 28 different target antigens specifically recognized by the IgGs of at least 20% of the patients suffering from Wegener's granulomatosis, 15 protein spots corresponding to 14 target antigens specifically recognized by the patients suffering from microscopic polyangiitis without anti-MPO Abs, five target antigens specifically recognized by the patients suffering from microscopic polyangiitis with anti-MPO Abs, 15 protein spots corresponding to 10 target antigens specifically recognized by the patients suffering from Churg-Strauss syndrome without anti-MPO Abs, and seven target antigens specifically recognized by the patients suffering from Churg-Strauss syndrome with anti-MPO Abs.

The detailed results are given in tables 1 to 5 below.

TABLE 1
Target antigens of AECAs of patients suffering from Wegener's granulomatosis

| | | | | | mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified^{#} | score | score | (%) |
| 228 | Vinculin | VINC_HUMAN | 124/116 | 5.5/6.6 | 3/13 | 53 | 34 | 15 |
| 382 | Far upstream element-binding protein 2 | FUBP2_HUMAN | 73/92 | 6.8/7.3 | 6/9 | 174 | 59 | 16 |
| 387 | Far upstream element-binding protein 2 | FUBP2_HUMAN | 73/91 | 6.8/7.1 | 2/9 | 90 | 53 | 17 |
| 438 | Caldesmon | CALD1_{—}HUMAN | 93/83 | 5.6/6.6 | 2/9 | 58 | 44 | 13 |
| 518 | 78 kDa glucose-regulated protein precursor | GRP78_{—}HUMAN | 72/75 | 5.1/5.4 | 13/21 | 1210 | 144 | 42 |
| 546 | Heat shock cognate 71 kDa protein | HSP7C_HUMAN | 71/75 | 5.4/5.9 | 9/13 | 284 | 73 | 29 |
| 575* | Heat shock cognate 71 kDa protein | HSP7C_{—}HUMAN | 71/75 | 5.4/5.7 | 9/10 | 308 | 77 | 21 |
| 579* | Stress protein 70 mitochondrial precursor | GRP75_HUMAN | 73/75 | 5.9/6.4 | 9/23 | 849 | 192 | 46 |
| 631 | Lamin-A/C | LMNA_HUMAN | 74/71 | 6.6/6.9 | 6/10 | 184 | 48 | 15 |
| 646 | Lamin-A/C | LMNA_HUMAN | 74/70 | 6.6/7 | 12/28 | 482 | 71 | 46 |
| 712* | Heat shock cognate 71 kDa protein | HSP7C_HUMAN | 71/64 | 5.4/5.4 | 8/13 | 298 | 53 | 26 |
| 738 | Heterogeneous nuclear ribonucleoprotein K | HNRPK_HUMAN | 51/62 | 5.4/5.8 | 6/10 | 224 | 54 | 31 |
| 740 | T-complex protein 1 subunit epsilon | TCPE_{—}HUMAN | 60/61 | 5.5/5.8 | 10/16 | 264 | 64 | 36 |
| 747* | 60 kDa heat shock protein mitochondrial | CH60_HUMAN | 61/62 | 5.7/5.6 | 7/7 | 509 | 123 | 21 |
| | precursor | | | | | | | |
| 776* | Protein disulfide isomerase A1 precursor | PDIA1_HUMAN | 57/59 | 4.8/5 | 10/20 | 769 | 119 | 52 |
| 797 | Protein disulfide isomerase A3 precursor | PDIA3_{—}HUMAN | 57/57 | 6/6.3 | 10/15 | 782 | 163 | 38 |
| 809 | Protein disulfide isomerase A3 precursor | PDIA3_HUMAN | 57/56 | 6/6.1 | 12/15 | 785 | 145 | 40 |
| 813 | T-complex protein 1 subunit theta | TCPQ_HUMAN | 60/57 | 5.4/6 | 2/2 | 67 | 33 | 3 |
| 814 | Protein disulfide isomerase A3 precursor | PDIA3_HUMAN | 57/56 | 6/6.0 | 6/14 | 318 | 80 | 36 |
| 820 | T-complex protein 1 subunit beta | TCPB_HUMAN | 57/55 | 6/6.6 | 11/12 | 421 | 92 | 38 |
| 821 | T-complex protein 1 subunit theta | TCPB_HUMAN | 57/56 | 6/6.6 | 12/17 | 608 | 89 | 51 |
| 844 | ATP synthase subunit alpha mitochondrial | ATPA_HUMAN | 60/54 | 9.2/9.3 | 7/12 | 288 | 75 | 32 |
| | precursor | | | | | | | |
| 874* | Heterogeneous nuclear ribonucleoprotein H | HNRH1_HUMAN | 49/52 | 5.9/6.4 | 9/12 | 649 | 124 | 40 |
| 910* | Tubulin beta-chain | TBB5_HUMAN | 50/50 | 4.8/5.4 | 11/18 | 578 | 91 | 56 |
| 966* | Fructose-bisphosphate aldolase A | ALDOA_HUMAN | 39/50 | 8.3/9.7 | 2/2 | 68 | 50 | 6 |
| 1031* | ATP synthase subunit alpha mitochondrial | ATPA_HUMAN | 60/47 | 9.2/9.5 | 5/6 | 276 | 96 | 15 |
| | precursor | | | | | | | |
| 1050* | Calumenin precursor | CALU_{—}HUMAN | 37/46 | 4.5/5.0 | 2/2 | 51 | 35 | 5 |
| 1113 | Reticulocalbin-3 | RCN3_HUMAN | 37/43 | 4.7/5.1 | 4/9 | 147 | 60 | 49 |
| 1165 | 26S proteasome non-ATPase regulatory subunit | PSD13_HUMAN | 43/40 | 5.5/6.3 | 4/6 | 59 | 27 | 18 |
| | 13 | | | | | | | |
| 1328 | Inorganic pyrophosphatase | IPYR_HUMAN | 33/34 | 5.5/6 | 9/11 | 359 | 82 | 47 |
| 1359 | Annexin A5 | ANXA5_HUMAN | 36/33 | 4.9/5.3 | 10/12 | 538 | 86 | 52 |
| 1480 | 14-3-3 protein epsilon | 1433E_HUMAN | 29/28 | 4.6/5.2 | 4/4 | 276 | 115 | 26 |
| 1514* | 6-phosphogluconolactonase | 6PGL_HUMAN | 28/26 | 5.7/6.1 | 6/7 | 214 | 62 | 36 |
| 1860* | Galectin-1 | LEG1_HUMAN | 15/15 | 5.3/5.3 | 4/4 | 156 | 49 | 34 |
| 2137 | Caldesmon | CALD1_HUMAN | 93/75 | 5.6/6.9 | 2/2 | 113 | 69 | 4 |
| 2151* | Succinyl-CoA: 3-keto acid-coenzyme A | SCOT_HUMAN | 56/60 | 7.1/6.8 | 5/6 | 200 | 68 | 21 |
| | transferase 1 mitochondrial precursor | | | | | | | |
| 2161* | Heterogeneous nuclear ribonucleoprotein D0 | HNRPD_HUMAN | 38/46 | 7.6/9.3 | 3/3 | 158 | 77 | 11 |

^{#}MSMS and MS + MSMS

All the spots in this table are recognized by the patients suffering from Wegener's granulomatosis and by the healthy individuals, the spots with a star are recognized specifically by the patients suffering from Wegener's granulomatosis and not by the patients suffering from other types of vasculitis. The antigens in bold are recognized by the serum IgGs of more than 60% of the pools of three sera of patients suffering from Wegener's granulomatosis.

TABLE 2
Target antigens of AECAs in the patients suffering from microscopic polyangiitis with anti-MPO ANCAs

| | | | | | Mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified^{#} | score | score | (%) |
| 851 | 26S protease regulatory subunit 7 | PRS7_HUMAN | 49/50 | 5.7/6.3 | 10/14 | 306 | 68 | 36 |
| 2077 | Heme oxygenase 2 | HMOX2_HUMAN | 36/36 | 5.3/5.8 | 2/7 | 77 | 54 | 35 |
| 2088 | Histone H2B type F-S | H2BFS_HUMAN | 14/22 | 10.4/5.8 | 2/4 | 77 | 60 | 33 |
| 2128 | Proteasome subunit alpha type-5 | PSA5_HUMAN | 26/34 | 4.7/5.7 | 4/5 | 210 | 91 | 24 |
| 2143 | Proteaseome subunit beta type-2 | PSB2_HUMAN | 23/32 | 6.5/7.1 | 5/5 | 236 | 68 | 32 |

TABLE 3
Target antigens of AECAs in the patients suffering from Churg-Strauss syndrome with anti-MPO ANCAs

| | | | | | Mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified ^{#} | score | score | (%) |
| 671 | Cytoskeleton-associated protein 4 | CKAP4_HUMAN | 5.6/5.2 | 66/67 | 3/6 | 80 | 32 | 12 |
| 1123 | Uroporphyrinogen decarboxylase | DCUP_HUMAN | 41/43 | 5.8/5.7 | 3/3 | 45 | 16 | 7 |
| 1669 | Adenine phosphoribosyltransferase | APT_HUMAN | 20/20 | 5.8/5.9 | 6/6 | 251 | 62 | 55 |
| 1836 | Profilin-1 | PROF1_HUMAN | 15/15 | 8.4/8.3 | 5/9 | 284 | 78 | 72 |
| 2083 | Plastin-3 | PLST_HUMAN | 70/73 | 5.5/6.3 | 9/17 | 388 | 91 | 34 |
| 2146 | Growth factor receptor-bound protein 2 | GRB2_HUMAN | 25/25 | 5.9/6.4 | 5/7 | 104 | 37 | 32 |
| 2152 | Heterogeneous nuclear ribonucleoprotein L | HNRPL_HUMAN | 64/69 | 8.5/7.3 | 4/10 | 52 | 19 | 24 |

TABLE 4
Target antigens of AECAs in the patients suffering from Churg-Strauss syndrome without anti-MPO ANCAs

| | | | | | Mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified ^{#} | score | score | (%) |
| 370 | Far upstream element-binding protein 2 | FUBP2_HUMAN | 73/91 | 6.8/7.1 | 4/8 | 151 | 64 | 16 |
| 382 | Far upstream element-binding protein 2 | FUBP2_HUMAN | 73/91 | 6.8/7.1 | 6/9 | 174 | 59 | 16 |
| 382 | Far upstream element-binding protein 2 | FUBP2_HUMAN | 73/92 | 6.8/7.3 | 6/9 | 174 | 59 | 16 |
| 387 | Far upstream element-binding protein 2 | FUBP2_HUMAN | 73/91 | 6.8/7.1 | 2/9 | 90 | 53 | 17 |
| 387 | Far upstream element-binding protein 2 | FUBP2_HUMAN | 73/91 | 6.8/7.1 | 2/9 | 90 | 53 | 17 |
| 1049 | Reticulocalbin-1 precursor | RCN1_HUMAN | 39/46 | 4.9/4.4 | 5/8 | 143 | 98 | 30 |
| 1077 | Calumenin precursor | CALU_{—}HUMAN | 37/45 | 4.5/4.4 | 6/10 | 148 | 32 | 41 |
| 1105 | SerpinB9 | SPB9_HUMAN | 42/43 | 5.6/6.2 | 6/12 | 182 | 64 | 31 |
| 1281 | Isocitrate dehydrogenase [NAD] subunit alpha | IDH3A_HUMAN | 40/37 | 6.5/6.1 | 3/5 | 119 | 59 | 15 |
| | mitochondrial precursor | | | | | | | |
| 1808 | Profilin-1 | PROF1_HUMAN | 15/15 | 8.4/8.9 | 2/3 | 39 | 23 | 27 |
| 2108 | GMP synthase [glutamine-hydrolyzing] | GUAA_HUMAN | 77/75 | 6.4/7.1 | 7/11 | 172 | 45 | 23 |
| 2132 | T-complex protein 1 subunit zeta | TCPZ_HUMAN | 58/88 | 6.2/7.5 | 2/3 | 39 | 20 | 6 |
| 2137 | Caldesmon | CALD1_{—}HUMAN | 93/75 | 5.6/6.9 | 2/2 | 113 | 69 | 4 |
| 2141 | Cofilin-1 | COF1_HUMAN | 18/17 | 8.2/8.2 | 4/6 | 242 | 68 | 40 |

* the antigens in bold are also recognized by the serum IgGs of more than 60% of the pools of three sera of patients suffering from Wegener's granulomatosis

TABLE 5
Target antigens of AECAs in the patients suffering from microscopic polyangiitis without anti-MPO ANCAs

| | | | | | Mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified ^{#} | score | score | (%) |
| 359 | Aconitate hydratase mitochondrial precursor | ACON_HUMAN | 85/96 | 7.4/7.6 | 10/18 | 385 | 56 | 31 |
| 457 | Mitochondrial inner membrane protein | IMMT_HUMAN | 84/81 | 6.1/7.4 | 4/8 | 85 | 40 | 14 |
| 497 | Heat shock cognate 71 kDa protein | HSP7C_HUMAN | 71/76 | 5.4/6.2 | 4/9 | 68 | 31 | 20 |
| 741 | Heterogeneous nuclear ribonucleoprotein K | HNRPK_HUMAN | 51/62 | 5.4/6.2 | 7/14 | 298 | 75 | 39 |
| 994 | Elongation factor Tu mitochondrial precursor | EFTU_HUMAN | 50/50 | 7.3/7.2 | 6/8 | 218 | 60 | 25 |
| 1033 | Alcohol dehydrogenase [NADP+] | AK1A1_HUMAN | 37/47 | 6.3/6.0 | 5/5 | 175 | 66 | 18 |
| 1156 | Alcohol dehydrogenase [NADP+] | AK1A1_HUMAN | 37/41 | 6.3/7.0 | 4/4 | 125 | 54 | 14 |
| 1171 | Sialic acid synthase | SIAS_HUMAN | 40/40 | 6.3/7.1 | 6/7 | 214 | 61 | 31 |
| 1391 | S-formylglutathione hydrolase | ESTD_HUMAN | 31/31 | 6.5/6.9 | 6 | 167 | 53 | 39 |
| 1394 | Guanine nucleotide-binding protein subunit | GBLP_HUMAN | 35/31 | 7.6/5.3 | 2/2 | 94 | 67 | 5 |
| | beta-2-like 1 | | | | | | | |
| 1398 | Purine nucleoside phosphorylase | PNPH_HUMAN | 32/31 | 6.5/6.6 | 6/7 | 363 | 106 | 32 |
| 1439 | Prohibitin | PHB_HUMAN | 30/29 | 5.6/6 | 9/13 | 451 | 75 | 64 |
| 1505 | C1q-binding protein mitochondrial precursor | C1QBP_HUMAN | 31/27 | 4.7/6.4 | 3/4 | 99 | 45 | 29 |
| 2130 | Transitional endoplasmic reticulum ATPase | TERA_HUMAN | 89/75 | 5.1/6.1 | 5/12 | 144 | 57 | 20 |
| 2162 | Nucleoside diphosphate kinase A | NDKA_HUMAN | 17/16 | 5.8/6.2 | 4/5 | 170 | 81 | 36 |

^{#} MSMS and MS + MSMS

### Example 2

### Characterization of the Antigenic Targets of The AECAs in Horton's Disease

The targets of the AECAs in the sera of 9 patients with Horton's disease, and of 12 healthy individuals, and pools of sera of patients suffering from thrombotic microangiopathy (4 pools of three) or from vasculitis (microscopic polyangiitis—4 pools of three, Wegener's disease—5 pools of three, and Churg-Strauss disease—3 pools of three) were investigated.

The serum IgG reactivities were analyzed by means of two-dimensional electrophoresis gels followed by immunoblotting using the endothelial cell antigens of HUVECs, as described in example 1.

The serum IgGs of patients suffering from Horton's disease recognized 162±3 protein spots in HUVEC extracts, while those of the healthy individuals recognized 79 protein spots. 28 protein spots were recognized by at least ⅔ of the pools of patients suffering from Horton's disease and not by the healthy individuals, of which 15 were identified. 26 HUVEC protein spots were recognized by at least one pool of sera of patients suffering from Horton's disease and not by the control sera nor by those of the healthy individuals, of which 9 were identified.

The detailed results are given in tables 6 and 7.

TABLE 6
Protein spots recognized by at least ⅔ of the pools of sera of patients
suffering from Horton's disease, and not recognized by the healthy individuals

| | | | | | Mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified ^{#} | score | score | (%) |
| 557 | Far upstream element-binding protein 1 | FUBP1_HUMAN | 67/75 | 7.2/7.2 | 3/7 | 114 | 47 | 13 |
| 631 | Lamin-A/C | LMNA_HUMAN | 74/71 | 6.6/6.9 | 6/10 | 184 | 48 | 15 |
| 646 | Lamin-A/C | LMNA_HUMAN | 74/70 | 6.6/7 | 12/28 | 482 | 71 | 46 |
| 784 | Dihydrolipoyl dehydrogenase mitochondrial | DLDH_HUMAN | 54/59 | 7.6/7.3 | 2/2 | 42 | 22 | 5 |
| | precursor | | | | | | | |
| 789 | Inosine-5′-monophosphate dehydrogenase 2 | IMDH2_HUMAN | 56/58 | 6.4/7.1 | 4/7 | 169 | 94 | 17 |
| 950 | Tripeptidyl-peptidase 1 precursor | TPP1_HUMAN | 61/50 | 6/6.4 | 3/5 | 89 | 34 | 15 |
| 1017 | Fumarate hydratase mitochondrial precursor | FUMH_HUMAN | 55/48 | 8.9/8 | 6/7 | 243 | 71 | 24 |
| 1085 | Heterogeneous nuclear ribonucleoprotein D0 | HNRPD_HUMAN | 38/43 | 7.6/7.8 | 3/3 | 122 | 69 | 11 |
| 1214 | PDZ and LIM domain protein 1 | PDLI1_HUMAN | 36/37 | 6.6/7.4 | 5/10 | 269 | 62 | 44 |
| 1249 | 60S acidic ribosomal protein P0 | RLA0_HUMAN | 34/37 | 5.7/6 | 2/5 | 56 | 35 | 21 |
| 1352 | Voltage-dependent anion-selective channel | VDAC2_HUMAN | 32/33 | 7.5/7.4 | 4/4 | 155 | 75 | 18 |
| | protein 2 | | | | | | | |
| 1359 | Annexin A5 | ANXA5_HUMAN | 36/33 | 4.9/5.3 | 10/12 | 538 | 86 | 52 |
| 1614 | DJ-1 protein | PARK7_HUMAN | 20/25 | 6.3/6.6 | 5/5 | 202 | 75 | 51 |
| 1734 | Peptidyl-prolyl cis-trans isomerase A | PPIA_HUMAN | 18/18 | 7.7/8 | 3/5 | 78 | 48 | 36 |
| 1817 | Thioredoxin-dependent peroxide reductase | PRDX3_HUMAN | 28/15 | 7.7/6.8 | 5/6 | 211 | 61 | 44 |
| | mitochondrial precursor | | | | | | | |

^{#} number of unique peptides identified during MSMS and MS + MSMS investigations

TABLE 7
Protein spots recognized by at least one pool of sera of patients suffering from Horton's disease, not recognized by the
healthy individuals, nor by the individuals suffering from other types of vasculitis or from thrombotic microangiopathies

| | | | | | Mass spectrometry | | | |
|---|---|---|---|---|---|---|---|---|
| | | | | | Number of | | | |
| | | SwissProt | Theoretical/ | Theoretical/ | unique | Total | Best | Sequence |
| N° on | | accession | estimated | estimated | peptides | ion | ion | coverage |
| the gel | Protein | number | MW (kDa) | PI | identified ^{#} | score | score | (%) |
| 228 | Vinculin | VINC_HUMAN | 124/116 | 5.5/6.6 | 3/13 | 53 | 34 | 15 |
| 407 | Far upstream element-binding protein 2 | FUBP2_HUMAN | 73/89 | 6.8/7.9 | 5/11 | 86 | 33 | 17 |
| 820 | T-complex protein 1 subunit beta | TCPB_HUMAN | 57/55 | 6/6.6 | 11/12 | 421 | 92 | 38 |
| 1115 | DnaJ homolog subfamily B member 11 precursor | DJB11_HUMAN | 40/43 | 5.8/6.5 | 7/8 | 316 | 116 | 34 |
| 1174 | Glutaredoxin-3 | GLRX3_HUMAN | 37/39 | 5.3/5.9 | 4/5 | 173 | 69 | 18 |
| 1328 | Inorganic pyrophosphatase | IPYR_HUMAN | 33/34 | 5.5/6 | 9/11 | 359 | 82 | 47 |
| 1493 | Rho GDP-dissociation inhibitor 2 | GDIR2_HUMAN | 23/27 | 5.1/5.7 | 5/10 | 246 | 77 | 58 |
| 1607 | Glutathione S-transferase P | GSTP1_HUMAN | 23/25 | 5.4/6 | 8/10 | 602 | 117 | 63 |
| 1633 | Peroxiredoxin-2 | PRDX2_HUMAN | 22/23 | 5.7/6 | 6/10 | 367 | 95 | 50 |

^{#} number of unique peptides identified during MSMS and MS + MSMS investigations

### Example 3

### Characterization of the Antigenic Targets of Anti-Vascular Smooth Muscle Cell Antibodies in Horton's Disease and Associated Types of Vasculitis

The sera of 15 patients suffering from Horton's disease (HD) and of 33 patients suffering from ANCA-associated vasculitis (15 having Wegener's granulomatosis GW, 9 having microscopic polyangiitis MPA, 9 having Churg-Strauss syndrome CSS) were tested in pools of three and compared with a pool of sera of 12 healthy individuals. The serum IgG reactivities were analyzed by means of two-dimensional electrophoresis gels followed by immunoblotting, virtually as described in example 1, but using antigens of mammary artery-derived immortalized vascular smooth muscle cells (VSMCs).

The serum IgGs of the pools of three patients suffering from Horton's disease (n=5), from GW with anti-proteinase 3 (PR3) ANCAs (n=5), from MPA with or without anti-myeloperoxidase (n=3), and from CSS with or without anti-MPO ANCAs (n=3) recognize 89±28, 94±34, 56±12 and 42±16 protein spots, respectively. Several antigens were specifically recognized by at least 60% of the groups of patients, and other antigens were recognized more strongly by the patients than by the healthy individuals.

The detailed results are given in tables 8 and 9.

TABLE 8
Antigens specifically recognized by the patients

| | | | | Number of | | | |
|---|---|---|---|---|---|---|---|
| | | Theoretical/ | | unique | Total | Best | Sequence |
| | SwissProt | estimated MW | Theoretical/ | peptides | ion | ion | coverage |
| Protein | number | (kDA) | estimated PI | identified # | score | score | (%) |
| Vinculin | VINC_HUMAN | 122/124 | 5.5/6.4 | 14 | 39 | | |
| Putative heat shock protein HSP 90-alpha A2 | HS902_HUMAN | 39/94 | 4.6/5.6 | 5 | 40 | | |
| Far upstream element-binding protein 2 | FUBP2_HUMAN | 73/88 | 6.8/7.2 | 4/10-9 | 67-46 | 24 | 21 |
| Far upstream element-binding protein 2 | FUBP2_HUMAN | 73/88 | 6.8/7.2 | 5/12-11 | 84-119 | 24 | 25 |
| Far upstream element-binding protein 2 | 3FUBP2_HUMAN | 73/88 | 6.8/7.8 | 11 | 85 | | |
| Lamin-A/C | LMNA_HUMAN | 74/67 | 6.6/6.5 | 10 | 24 | | |
| Coatomer subunit alpha | COPA_HUMAN | 138/67 | 7.7/6.5 | 3 | 37 | | |
| UDP-glucose 6-dehydrogenase | UGDH_HUMAN | 55/66 | 6.6/6.7 | 4 | 46 | | |
| Protein disulfide isomerase A3 | PDIA3_HUMAN | 57/59 | 6.7/7.5 | 12 and 16 | 528-804 | | |
| Protein disulfide isomerase A3 | PDIA3_HUMAN | 57/59 | 6.0/6.3 | 14-13 | 460-362 | | |
| T-complex protein 1 subunit beta | TCPB_HUMAN | 57/57 | 6.0/6.5 | 14-14 | 503-519 | | |
| Actin, cytoplasmic 1 | ACTB_HUMAN | 42/47 | 5.3/5.7 | 5/10-9 | 390-418 | 131 | 53 |
| POTE ankyrin domain family member E | POTEE_HUMAN | 121/47 | 5.8/5.7 | 6 | 251 | | |
| Nucleophosmin | NPM_HUMAN | 33/39 | 4.6/5.1 | 3/5-5 | 83-203 | 39 | 24 |
| Annexin A2 | ANXA2_HUMAN | 39/35 | 7.6/8.0 | 13 and 10 | 650-265 | | |

# number of unique peptides identified during MSMS and MS + MSMS investigations

TABLE 9
Antigens recognized more strongly by the patients than by the healthy individuals

| | | | | Number of | | | |
|---|---|---|---|---|---|---|---|
| | | Theoretical/ | | unique | Total | Best | Sequence |
| | SwissProt | estimated MW | Theoretical/ | peptides | ion | ion | coverage |
| Protein | number | (kDA) | estimated PI | identified # | score | score | (%) |
| Elongation factor 2 | EF2_HUMAN | 95/101 | 6.4/7.4 | 16 | 198 | | |
| Caldesmon | CALD1_HUMAN | 93/84 | 5.6/6.7 | 3/6-8 | 72-288 | 39 | 9 |
| Inosine-5′-monophosphate dehydrogenase 2 | IMDH2_HUMAN | 56/60 | 6.4/7.1 | 14 | 474 | | |
| Alpha-enolase | ENOA_HUMAN | 47/57 | 7.0/7.7 | 7/12-7 | 245-66 | 72 | 44 |
| DnaJ homolog subfamily A member 1 | DNJA1_HUMAN | 45/49 | 6.7/7.4 | 9 | 58 | | |
| Actin, cytoplasmic 2 | ACTG_HUMAN | 42/45 | 5.3/5.5 | 5/7-13 | 258-430 | 116 | 34 |
| 26S protease regulatory subunit 8 | PRS8_HUMAN | 46/46 | 7.1/7.6 | 15 | 180 | | |

# number of unique peptides identified during MSMS and MS + MSMS investigations

1. An in vitro method for detecting vasculitis chosen from Wegener's granulomatosis, microscopic polyangiitis, Churg-Strauss syndrome and Horton's disease, in an individual, or the risk of developing said vasculitis, which comprises determining the presence and/or the amount of at least one anti-endothelial cell antibody (AECA) or anti-vascular smooth muscle cell (VSMC) antibody.

2. The method as claimed in claim 1, for detecting Wegener's granulomatosis, or the risk of developing Wegener's granulomatosis, which comprises determining the presence and/or the amount of at least one antibody directed against an antigen chosen from the group consisting of vinculin, FUbp2 (far upstream element-binding protein 2), caldesmon, 78 kDa glucose-regulating protein precursor, heat shock cognate 71 kDa protein, stress protein 70 mitochondrial precursor, lamin-A/C, heterogeneous nuclear ribonucleoprotein K, T-complex protein 1 subunit epsilon, 60 kDa heat shock protein mitochondrial precursor, protein disulfide isomerase A1 precursor, protein disulfide isomerase A3 precursor, T-complex protein 1 subunit theta, T-complex protein 1 subunit beta, ATP synthase subunit alpha mitochondrial precursor, heterogeneous nuclear ribonucleoprotein H, tubulin beta-chain, fructose-bisphosphate aldolase A, ATP synthase subunit alpha mitochondrial precursor, calumenin precursor, reticulocalbin-3, 26S proteasome non-ATPase regulatory subunit 13, inorganic pyrophosphatase, annexin A5, 14-3-3 protein epsilon, 6-phosphogluconolactonase, galectin-1, succinyl-CoA:3-keto acid-conenzyme A transferase 1 mitochondrial precursor and heterogeneous nuclear ribonucleoprotein D0, in a biological sample from a patient, the presence of said at least one antibody being an indicator of Wegener's granulomatosis or of the risk of developing Wegener's granulomatosis.

3. The method as claimed in claim 1, for detecting microscopic polyangiitis, or the risk of developing microscopic polyangiitis, which comprises determining the presence and/or the amount of at least one antibody directed against an antigen chosen from the group consisting of 26S protease regulatory subunit 7, heme oxygenase 2, histone H2B type F-S, proteasome subunit alpha type-5, proteasome subunit beta type-2, aconitate hydratase mitochondrial precursor, mitochondrial inner membrane protein, heterogeneous nuclear ribonucleoprotein K, elongation factor Tu mitochondrial precursor, alcohol dehydrogenase [NADP+], sialic acid synthase, S-formylglutathione hydrolase, guanine nucleotide-binding protein subunit beta-2-like 1, purine nucleoside phosphorylase, prohibitin, C1q-binding protein mitochondrial precursor, transitional endoplasmic reticulum ATPase and nucleoside diphosphate kinase A, in a biological sample from a patient, the presence of said at least one antibody being an indicator of microscopic polyangiitis or of the risk of developing microscopic polyangiitis.

4. The method as claimed in claim 1, for detecting Churg-Strauss syndrome, or the risk of developing Churg-Strauss syndrome, which comprises determining the presence and/or the amount of at least one antibody directed against an antigen chosen from the group consisting of cytoskeleton-associated protein 4, uroporphyrinogen decarboxylase, adenine phosphoribosyltransferase, profilin-1, plastin-3, growth factor receptor-bound protein 2, heterogeneous nuclear ribonucleoprotein L, FUbp2 (far upstream element-binding protein 2), reticulocalbin-1 precursor, calumenin precursor, serpinB9, isocitrate dehydrogenase [NAD] subunit alpha mitochondrial precursor, GMP synthase [glutamine-hydrolyzing], T-complex protein 1 subunit zeta, caldesmon and cofilin-1, in a biological sample from a patient, the presence of said at least one antibody being an indicator of Churg-Strauss syndrome or of the risk of developing Churg-Strauss syndrome.

5. The method as claimed in claim 1, for detecting Horton's disease, or the risk of developing Horton's disease, which comprises determining the presence and/or the amount of at least one antibody directed against an antigen chosen from the group consisting of vinculin, caldesmon, lamin-A/C, alpha-enolase, actin, nucleophosmin, annexin A2, FUbp2 (far upstream element-binding protein 2), FUbp1 (far upstream element-binding protein 1), dihydrolipoyl dehydrogenase mitochondrial precursor, inosine-5′-monophosphate dehydrogenase 2, tripeptidyl-peptidase 1 precursor, fumarate hydratase mitochondrial precursor, PDZ and LIM domain protein 1, 60S acidic ribosomal protein P0, voltage-dependent anion-selective channel protein 2, DJ-1 protein, peptidyl-prolyl cis-trans isomerase A, thioredoxin-dependent peroxide reductase mitochondrial precursor, T-complex protein 1 subunit beta, DNAJ homolog subfamily B member 11 precursor, glutaredoxin-3, inorganic pyrophosphatase, Rho GDP dissociation inhibitor protein 2 and glutathione S-transferase P, in a biological sample from a patient, the presence of said at least one antibody being an indicator of Horton's disease or of the risk of developing Horton's disease.

6. The method as claimed in claim 1, which comprises determining the presence and/or the amount of at least one antibody directed against vinculin or lamin.

7. The method as claimed in claim 1, which comprises determining the presence and/or the amount of at least one antibody directed against an antigen chosen from the group consisting of caldesmon, 78 kDa glucose-regulated protein precursor, heat shock cognate 71 kDa protein, T-complex protein 1 subunit epsilon, protein disulfide isomerase A3 precursor and calumenin precursor.

8. The method as claimed in claim 1, in which the biological sample is a blood or serum sample.

9. The method as claimed in claim 1, in which the presence of said at least one antibody in the biological sample is compared with a control value, the presence of said at least one antibody in an amount greater than the control value being an indicator of vasculitis or of the risk of developing vasculitis.

10. The method as claimed in claim 1, in which the amount of said at least one antibody is determined by means of an immunoassay.

11. The method as claimed in claim 10, in which the immunoassay is an ELISA assay.

12. The method as claimed in claim 1, in which the patient is a human being.

13. The method as claimed in claim 1, in which the patient does not have ANCA autoantibodies.

14. An in vitro method for the prognosis or monitoring of vasculitis chosen from Wegener's granulomatosis, microscopic polyangiitis, Churg-Strauss syndrome and Horton's disease, which comprises determining the presence and/or the amount of at least one antibody as defined in claim 1, in a biological sample from a patient, at various times, an increase in the amount of said at least one antibody over time being an indication of a worsening of the vasculitis.

15. An in vitro method for evaluating the efficacy of a treatment for vasculitis chosen from Wegener's granulomatosis, microscopic polyangiitis, Churg-Strauss syndrome and Horton's disease, which comprises determining the presence and/or the amount of at least one antibody as defined in claim 1, in a biological sample from a patient, at various times before, during or after the treatment, a decrease in the amount of said at least one antibody over time being an indication of an improvement of the vasculitis.

16. The method as claimed in claim 2, in which the biological sample is a blood or serum sample.

17. The method as claimed in claim 3, in which the biological sample is a blood or serum sample.

18. The method as claimed in claim 4, in which the biological sample is a blood or serum sample.

19. The method as claimed in claim 5, in which the biological sample is a blood or serum sample.

20. The method as claimed in claim 6, in which the biological sample is a blood or serum sample.

21. The method as claimed in claim 7, in which the biological sample is a blood or serum sample.


SEQUENCE LISTING (88 sequences)

SEQ ID NO: 1 - protein, 1134 aa, Homo sapiens
  MISC_FEATURE: Vinculin
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKPGIPAAEVGIGVVAEADAADAAGFPVPPDMEDDYEPELLLMPSNQPVNQPILAAAQSLHREATKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 2 - protein, 710 aa, Homo sapiens
  MISC_FEATURE: Far upstream element-binding protein 2
MSDYSTGGPPPGPPPPAGGGGGAGGAGGGPPPGPPGAGDRGGGGPCGGGPGGGSAGGPSQPPGGGGPGIRKDAFADAVQRARQIAAKIGGDAATTVNNSTPDFGFGGQKRQLEDGDQPESKKLASQGDSISSQLGPIHPPPRTSMTEEYRVPDGMVGLIIGRGGEQINKIQQDSGCKVQISPDSGGLPERSVSLTGAPESVQKAKMMLDDIVSRGRGGPPGQFHDNANGGQNGTVQEIMIPAGKAGLVIGKGGETIKQLQERAGVKMILIQDGSQNTNVDKPLRIIGDPYKVQQACEMVMDILRERDQGGFGDRNEYGSRIGGGIDVPVPRHSVGVVIGRSGEMIKKIQNDAGVRIQFKQDDGTGPEKIAHIMGPPDRCEHAARIINDLLQSLRSGPPGPPGGPGMPPGGRGRGRGQGNWGPPGGEMTFSIPTHKCGLVIGRGGENVKAINQQTGAFVEISRQLPPNGDPNFKLFIIRGSPQQIDHAKQLIEEKIEGPLCPVGPGPGGPGPAGPMGPFNPGPFNQGPPGAPPHAGGPPPHQYPPQGWGNTYPQWQPPAPHDPSKAAAAAADPNAAWAAYYSHYYQQPPGPVPGPAPAPAAPPAQGEPPQPPPTGQSDYTKAWEEYYKKIGQQPQQPGAPPQQDYTKAWEEYYKKQAQVATGGGPGAPPGSQPDYSAAWAEYYRQQAAYYGQTPVPGPQPPPTQQGQQQAQ

SEQ ID NO: 3 - protein, 710 aa, Homo sapiens; identical to SEQ ID NO: 2
  MISC_FEATURE: Caldesmon

SEQ ID NO: 4 - protein, 654 aa, Homo sapiens
  MISC_FEATURE: 78 kDa glucose-regulated protein precursor
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 5 - protein, 646 aa, Homo sapiens
  MISC_FEATURE: Heat shock cognate 71 kDa protein
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIINWLDKNQTAEKEEFEHQQKELEKVCNPIITKLYQSAGGMPGGMPGGFPGGGAPPSGGASSGPTIEEVD

SEQ ID NO: 6 - protein, 679 aa, Homo sapiens
  MISC_FEATURE: Stress protein 70 mitochondrial precursor
MISASRAAAARLVGAAASRGPTAARHQDSWNGLSHEAFRLVSRRDYASEAIKGAVVGIDLGTTNSCVAVMEGKQAKVLENAEGARTTPSVVAFTADGERLVGMPAKRQAVTNPNNTFYATKRLIGRRYDDPEVQKDIKNVPFKIVRASNGDAWVEAHGKLYSPSQIGAFVLMKMKETAENYLGHTAKNAVITVPAYFNDSQRQATKDAGQISGLNVLRVINEPTAAALAYGLDKSEDKVIAVYDLGGGTFDISILEIQKGVFEVKSTNGDTFLGGEDFDQALLRHIVKEFKRETGVDLTKDNMALQRVREAAEKAKCELSSSVQTDINLPYLTMDSSGPKHLNMKLTRAQFEGIVTDLIRRTIAPCQKAMQDAEVSKSDIGEVILVGGMTRMPKVQQTVQDLFGRAPSKAVNPDEAVAIGAAIQGGVLAGDVTDVLLLDVTPLSLGIETLGGVFTKLINRNTTIPTKKSQVFSTAADGQTQVEIKVCQGEREMAGDNKLLGQFTLIGIPPAPRGVPQIEVTFDIDANGIVHVSAKDKGTGREQQIVIQSSGGLSKDDIENMVKNAEKYAEEDRRKKERVEAVNMAEGIIHDTETKMEEFKDQLPADECNKLKEEISKMRELLARKDSETGENIRQAASSLQQASLKLFEMAYKKMASEREGSGSSGTGEQKEDQKEEKQ

SEQ ID NO: 7 - protein, 664 aa, Homo sapiens
  MISC_FEATURE: Lamin-A/C
METPSQRRATRSGAQASSTPLSPTRITRLQEKEDLQELNDRLAVYIDRVRSLETENAGLRLRITESEEVVSREVSGIKAAYEAELGDARKTLDSVAKERARLQLELSKVREEFKELKARNTKKEGDLIAAQARLKDLEALLNSKEAALSTALSEKRTLEGELHDLRGQVAKLEAALGEAKKQLQDEMLRRVDAENRLQTMKEELDFQKNIYSEELRETKRRHETRLVEIDNGKQREFESRLADALQELRAQHEDQVEQYKKELEKTYSAKLDNARQSAERNSNLVGAAHEELQQSRIRIDSLSAQLSQLQKQLAAKEAKLRDLEDSLARERDTSRRLLAEKEREMAEMRARMQQQLDEYQELLDIKLALDMEIHAYRKLLEGEEERLRLSPSPTSQRSRGRASSHSSQTQGGGSVTKKRKLESTESRSSFSQHARTSGRVAVEEVDEEGKFVRLRNKSNEDQSMGNWQIKRQNGDDPLLTYRFPPKFTLKAGQVVTIWAAGAGATHSPPTDLVWKAQNTWGCGNSLRTALINSTGEEVAMRKLVRSVTVVEDDEDEDGDDLLHHHHGSHCSSSGDPAEYNLRSRTVLCGTCGQPADKASASGSGAQVGGPISSGSSASSVTVTRSYRSVGGSGGGSFGDNLVTRSYLLGNSSPRTQSPQNCSIM

SEQ ID NO: 8 - protein, 463 aa, Homo sapiens
  MISC_FEATURE: Heterogeneous nuclear ribonucleoprotein K
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 9 - protein, 541 aa, Homo sapiens
  MISC_FEATURE: T-complex protein 1 subunit epsilon
MASMGTLAFDEYGRPFLIIKDQDRKSRLMGLEALKSHIMAAKAVANTMRTSLGPNGLDKMMVDKDGDVTVTNDGATILSMMDVDHQIAKLMVELSKSQDDEIGDGTTGVVVLAGALLEEAEQLLDRGIHPIRIADGYEQAARVAIEHLDKISDSVLVDIKDTEPLIQTAKTTLGSKVVNSCHRQMAEIAVNAVLTVADMERRDVDFELIKVEGKVGGRLEDTKLIKGVIVDKDFSHPQMPKKVEDAKIAILTCPFEPPKPKTKHKLDVTSVEDYKALQKYEKEKFEEMIQQIKETGANLAICQWGFDDEANHLLLQNNLPAVRWVGGPEIELIAIATGGRIVPRFSELTAEKLGFAGLVQEISFGTTKDKMLVIEQCKNSRAVTIFIRGGNKMIIEEAKRSLHDALCVIRNLIRDNRVVYGGGAAEISCALAVSQEADKCPTLEQYAMRAFADALEVIPMALSENSGMNPIQTMTEVRARQVKEMNPALGIDCLHKGTNDMKQQHVIETLIGKKQQISLATQMVRMILKIDDIRKPGESEE

SEQ ID NO: 10 - protein, 573 aa, Homo sapiens
  MISC_FEATURE: 60 kDa heat shock protein mitochondrial precursor
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 11 - protein, 508 aa, Homo sapiens
  MISC_FEATURE: Protein disulfide isomerase A1 precursor
MLRRALLCLAVAALVRADAPEEEDHVLVLRKSNFAEALAAHKYLLVEFYAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHSFPTLKFFPASADRTVIDYNGERTLDGFKKFLESGGQDGAGDDDDLEDLEEAEEPDMEEDDDQKAVKDEL

SEQ ID NO: 12 - protein, 505 aa, Homo sapiens
  MISC_FEATURE: Protein disulfide isomerase A3 precursor
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 13 - protein, 548 aa, Homo sapiens
  MISC_FEATURE: T-complex protein 1 subunit theta
MALHVPKAPGFAQMLKEGAKHFSGLEEAVYRNIQACKELAQTTRTAYGPNGMNKMVINHLEKLFVTNDAATILRELEVQHPAAKMIVMASHMQEQEVGDGTNFVLVFAGALLELAEELLRIGLSVSEVIEGYEIACRKAHEILPNLVCCSAKNLRDIDEVSSLLRTSIMSKQYGNEVFLAKLIAQACVSIFPDSGHFNVDNIRVCKILGSGISSSSVLHGMVFKKETEGDVTSVKDAKIAVYSCPFDGMITETKGTVLIKTAEELMNFSKGEENLMDAQVKAIADTGANVVVTGGKVADMALHYANKYNIMLVRLNSKWDLRRLCKTVGATALPRLTPPVLEEMGHCDSVYLSEVGDTQVVVFKHEKEDGAISTIVLRGSTDNLMDDIERAVDDGVNTFKVLTRDKRLVPGGGATEIELAKQITSYGETCPGLEQYAIKKFAEAFEAIPRALAENSGVKANEVISKLYAVHQEGNKNVGLDIEAEVPAVKDMLEAGILDTYLGKYWAIKLATNAAVTVLRVDQIIMAKPAGGPKPPSGKKDWDDDQND

SEQ ID NO: 14 - protein, 535 aa, Homo sapiens
  MISC_FEATURE: T-complex protein 1 subunit beta
MASLSLAPVNIFKAGADEERAETARLTSFIGAIAIGDLVKSTLGPKGMDKILLSSGRDASLMVTNDGATILKNIGVDNPAAKVLVDMSRVQDDEVGDGTTSVTVLAAELLREAESLIAKKIHPQTIIAGWREATKAAREALLSSAVDHGSDEVKFRQDLMNIAGTTLSSKLLTHHKDHFTKLAVEAVLRLKGSGNLEAIHIIKKLGGSLADSYLDEGFLLDKKIGVNQPKRIENAKILIANTGMDTDKIKIFGSRVRVDSTAKVAEIEHAEKEKMKEKVERILKHGINCFINRQLIYNYPEQLFGAAGVMAIEHADFAGVERLALVTGGEIASTFDHPELVKLGSCKLIEEVMIGEDKLIHFSGVALGEACTIVLRGATQQILDEAERSLHDALCVLAQTVKDSRTVYGGGCSEMLMAHAVTQLANRTPGKEAVAMESYAKALRMLPTIIADNAGYDSADLVAQLRAAHSEGNTTAGLDMREGTIGDMAILGITESFQVKRQVLLSAAEAAEVILRVDNIIKAAPRKRVPDHHPC

SEQ ID NO: 15 - protein, 553 aa, Homo sapiens
  MISC_FEATURE: ATP synthase subunit alpha mitochondrial precursor
MLSVRVAAAVVRALPRRAGLVSRNALGSSFIAARNFHASNTHLQKTGTAEMSSILEERILGADTSVDLEETGRVLSIGDGIARVHGLRNVQAEEMVEFSSGLKGMSLNLEPDNVGVVVFGNDKLIKEGDIVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 16 - protein, 449 aa, Homo sapiens
  MISC_FEATURE: Heterogeneous nuclear ribonucleoprotein H
MMLGTEGGEGFVVKVRGLPWSCSADEVQRFFSDCKIQNGAQGIRFIYTREGRPSGEAFVELESEDEVKLALKKDRETMGHRYVEVFKSNNVEMDWVLKHTGPNSPDTANDGFVRLRGLPFGCSKEEIVQFFSGLEIVPNGITLPVDFQGRSTGEAFVQFASQEIAEKALKKHKERIGHRYIEIFKSSRAEVRTHYDPPRKLMAMQRPGPYDRPGAGRGYNSIGRGAGFERMRRGAYGGGYGGYDDYNGYNDGYGFGSDRFGRDLNYCFSGMSDHRYGDGGSTFQSTTGHCVHMRGLPYRATENDIYNFFSPLNPVRVHIEIGPDGRVTGEADVEFATHEDAVAAMSKDKANMQHRYVELFLNSTAGASGGAYEHRYVELFLNSTAGASGGAYGSQMMGGMGLSNQSSYGGPASQQLSGGYGGGYGGQSSMSGYDQVLQENSSDFQSNIA

SEQ ID NO: 17 - protein, 444 aa, Homo sapiens
  MISC_FEATURE: Tubulin beta-chain
MREIVHIQAGQCGNQIGAKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEA

SEQ ID NO: 18 - protein, 364 aa, Homo sapiens
  MISC_FEATURE: Fructose-bisphosphate aldolase A
MPYQYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKFSHEEIAMATVTALRRTVPPAVTGITFLSGGQSEEEASINLNAINKCPLLKPWALTFSYGRALQASALKAWGGKKENLKAAQEEYVKRALANSLACQGKYTPSGQAGAAASESLFVSNHAY

SEQ ID NO: 19 - protein, 315 aa, Homo sapiens
  MISC_FEATURE: Calumenin precursor
MDLRQFLMCLSLCTAFALSKPTEKKDRVHHEPQLSDKVHNDAQSFDYDHDAFLGAEEAKTFDQLTPEESKERLGKIVSKIDGDKDGFVTVDELKDWIKFAQKRWIYEDVERQWKGHDLNEDGLVSWEEYKNATYGYVLDDPDPDDGFNYKQMMVRDERRFKMADKDGDLIATKEEFTAFLHPEEYDYMKDIVVQETMEDIDKNADGFIDLEEYIGDMYSHDGNTDEPEWVKTEREQFVEFRDKNRDGKMDKEETKDWILPSDYDHAEAEARHLVYESDQNKDGKLTKEEIVDKYDLFVGSQATDFGEALVRHDEF

SEQ ID NO: 20 - protein, 328 aa, Homo sapiens
  MISC_FEATURE: Reticulocalbin-3
MMWRPSVLLLLLLLRHGAQGKPSPDAGPHGQGRVHQAAPLSDAPHDDAHGNFQYDHEAFLGREVAKEFDQLTPEESQARLGRIVDRMDRAGDGDGWVSLAELRAWIAHTQQRHIRDSVSAAWDTYDTDRDGRVGWEELRNATYGHYAPGEEFHDVEDAETYKKMLARDERRFRVADQDGDSMATREELTAFLHPEEFPHMRDIVIAETLEDLDRNKDGYVQVEEYIADLYSAEPGEEEPAWVQTERQQFRDFRDLNKDGHLDGSEVGHWVLPPAQDQPLVEANHLLHESDTDKDGRLSKAEILGNWNMFVGSQATNYGEDLTRHHDEL

SEQ ID NO: 21 - protein, 376 aa, Homo sapiens
  MISC_FEATURE: 26S proteasome non-ATPase regulatory subunit 13
MKDVPGFLQQSQSSGPGQPAVWHRLEELYTKKLWHQLTLQVLDFVQDPCFAQGDGLIKLYENFISEFEHRVNPLSLVEIILHVVRQMTDPNVALTFLEKTREKVKSSDEAVILCKTAIGALKLNIGDLQVTKETIEDVEEMLNNLPGVTSVHSRFYDLSSKYYQTIGNHASYYKDALRFLGCVDIKDLPVSEQQERAFTLGLAGLLGEGVFNFGELLMHPVLESLRNTDRQWLIDTLYAFNSGNVERFQTLKTAWGQQPDLAANEAQLLRKIQLLCLMEMTFTRPANHRQLTFEEIAKSAKITVNEVELLVMKALSVGLVKGSIDEVDKRVHMTWVQPRVLDLQQIKGMKDRLEFWCTDVKSMEMLVEHQAHDILT

SEQ ID NO: 22 - protein, 289 aa, Homo sapiens
  MISC_FEATURE: Inorganic pyrophosphatase
MSGFSTEERAAPFSLEYRVFLKNEKGQYISPFHDIPIYADKDVFHMVVEVPRWSNAKMEIATKDPLNPIKQDVKKGKLRYVANLFPYKGYIWNYGAIPQTWEDPGHNDKHTGCCGDNDPIDVCEIGSKVCARGEIIGVKVLGILAMIDEGETDWKVIAINVDDPDAANYNDINDVKRLKPGYLEATVDWFRRYKVPDGKPENEFAFNAEFKDKDFAIDIIKSTHDHWKALVTKKTNGKGISCMNTTLSESPFKCDPDAARAIVDALPPPCESACTVPTDVDKWFHHQKN

SEQ ID NO: 23 - protein, 320 aa, Homo sapiens
  MISC_FEATURE: Annexin A5
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 24 - protein, 255 aa, Homo sapiens
  MISC_FEATURE: 14-3-3 protein epsilon
MDDREDLVYQAKLAEQAERYDEMVESMKKVAGMDVELTVEERNLLSVAYKNVIGARRASWRIISSIEQKEENKGGEDKLKMIREYRQMVETELKLICCDILDVLDKHLIPAANTGESKVFYYKMKGDYHRYLAEFATGNDRKEAAENSLVAYKAASDIAMTELPPTHPIRLGLALNFSVFYYEILNSPDRACRLAKAAFDDAIAELDTLSEESYKDSTLIMQLLRDNLTLWTSDMQGDGEEQNKEALQDVEDENQ

SEQ ID NO: 25 - protein, 258 aa, Homo sapiens
  MISC_FEATURE: 6-phosphogluconolactonase
MAAPAPGLISVFSSSQELGAALAQLVAQRAACCLAGARARFALGLSGGSLVSMLARELPAAVAPAGPASLARWTLGFCDERLVPFDHAESTYGLYRTHLLSRLPIPESQVITINPELPVEEAAEDYAKKLRQAFQGDSIPVFDLLILGVGPDGHTCSLFPDHPLLQEREKIVAPISDSPKPPPQRVTLTLPVLNAARTVIFVATGEGKAAVLKRILEDQEENPLPAALVQPHTGKLCWFLDEAAARLLTVPFEKHSTL

SEQ ID NO: 26 - protein, 135 aa, Homo sapiens
  MISC_FEATURE: Galectin-1
MACGLVASNLNLKPGECLRVRGEVAPDAKSFVLNLGKDSNNLCLHFNPRFNAHGDANTIVCNSKDGGAWGTEQREAVFPFQPGSVAEVCITFDQANLTVKLPDGYEFKFPNRLNLEAINYMAADGDFKIKCVAFD

SEQ ID NO: 27 - protein, 520 aa, Homo sapiens
  MISC_FEATURE: Succinyl-CoA3-keto acid-coenzyme A transferase 1 mitochondrial precursor
MAALKLLSSGLRLCASARGSGATWYKGCVCSFSTSAHRHTKFYTDPVEAVKDIPDGATVLVGGFGLCGIPENLIDALLKTGVKGLTAVSNNAGVDNFGLGLLLRSKQIKRMVSSYVGENAEFERQYLSGELEVELTPQGTLAERIRAGGAGVPAFYTPTGYGTLVQEGGSPIKYNKDGSVAIASKPREVREFNGQHFILEEAITGDFALVKAWKADRAGNVIFRKSARNFNLPMCKAAETTVVEVEEIVDIGAFAPEDIHIPQIYVHRLIKGEKYEKRIERLSIRKEGDGEAKSAKPGDDVRERIIKRAALEFEDGMYANLGIGIPLLASNFISPNITVHLQSENGVLGLGPYPRQHEADADLINAGKETVTILPGASFFSSDESFAMIRGGHVDLTMLGAMQVSKYGDLANWMIPGKMVKGMGGAMDLVSSAKTKVVVTMEHSAKGNAHKIMEKCTLPLTGKQCVNRIITEKAVFDVDKKKGLTLIELWEGLTVDDVQKSTGCDFAVSPKLMPMQQIAN

SEQ ID NO: 28 - protein, 355 aa, Homo sapiens
  MISC_FEATURE: Heterogeneous nuclear ribonucleoprotein D0
MSEEQFGGDGAAAAATAAVGGSAGEQEGAMVAATQGAAAAAGSGAGTGGGTASGGTEGGSAESEGAKIDASKNEEDEGHSNSSPRHSEAATAQREEWKMFIGGLSWDTTKKDLKDYFSKFGEVVDCTLKLDPITGRSRGFGFVLFKESESVDKVMDQKEHKLNGKVIDPKRAKAMKTKEPVKKIFVGGLSPDTPEEKIREYFGGFGEVESIELPMDNKTNKRRGFCFITFKEEEPVKKIMEKKYHNVGLSKCEIKVAMSKEQYQQQQQWGSRGGFAGRARGRGGGPSQNWNQGYSNYWNQGYGNYGYNSQGYGGYGGYDYTGYNNYYGYGDYSNQQSGYGKVSRRGGHQNSYKPY

SEQ ID NO: 29 - protein, 433 aa, Homo sapiens
  MISC_FEATURE: 26S protease regulatory subunit 7
MPDYLGADQRKTKEDEKDDKPIRALDEGDIALLKTYGQSTYSRQIKQVEDDIQQLLKKINELTGIKESDTGLAPPALWDLAADKQTLQSEQPLQVARCTKIINADSEDPKYIINVKQFAKFVVDLSDQVAPTDIEEGMRVGVDRNKYQIHIPLPPKIDPTVTMMQVEEKPDVTYSDVGGCKEQIEKLREVVETPLLHPERFVNLGIEPPKGVLLFGPPGTGKTLCARAVANRTDACFIRVIGSELVQKYVGEGARMVRELFEMARTKKACLIFFDEIDAIGGARFDDGAGGDNEVQRTMLELINQLDGFDPRGNIKVLMATNRPDTLDPALMRPGRLDRKIEFSLPDLEGRTHIFKIHARSMSVERDIRFELLARLCPNSTGAEIRSVCTEAGMFAIRARRKIATEKDFLEAVNKVIKSYAKFSATPRYMTYN

SEQ ID NO: 30 - protein, 316 aa, Homo sapiens
  MISC_FEATURE: Heme oxygenase 2
MSAEVETSEGVDESEKKNSGALEKENQMRMADLSELLKEGTKEAHDRAENTQFVKDFLKGNIKKELFKLATTALYFTYSALEEEMERNKDHPAFAPLYFPMELHRKEALTKDMEYFFGENWEEQVQCPKAAQKYVERIHYIGQNEPELLVAHAYTRYMGDLSGGQVLKKVAQRALKLPSTGEGTQFYLFENVDNAQQFKQLYRARMNALDLNMKTKERIVEEANKAFEYNMQIFNELDQAGSTLARETLEDGFPVHDGKGDMRKCPFYAAEQDKGALEGSSCPFRTAMAVLRKPSLQFILAAGVALAAGLLAWYYM

SEQ ID NO: 31 - protein, 126 aa, Homo sapiens
  MISC_FEATURE: Histone H2B type F-S
MPEPAKSAPAPKKGSKKAVTKAQKKDGRKRKRSRKESYSVYVYKVLKQVHPDTGISSKAMGIMNSFVNDIFERIAGEASRLPHYNKRSTITSREIQTAVRLLLPGELAKHAVSEGTKAVTKYTSAK

SEQ ID NO: 32 - protein, 241 aa, Homo sapiens
  MISC_FEATURE: Proteasome subunit alpha type-5
MFLTRSEYDRGVNTFSPEGRLFQVEYAIEAIKLGSTAIGIQTSEGVCLAVEKRITSPLMEPSSIEKIVEIDAHIGCAMSGLIADAKTLIDKARVETQNHWFTYNETMTVESVTQAVSNLALQFGEEDADPGAMSRPFGVALLFGGVDEKGPQLFHMDPSGTFVQCDARAIGSASEGAQSSLQEVYHKSMTLKEAIKSSLIILKQVMEEKLNATNIELATVQPGQNFHMFTKEELEEVIKDI

SEQ ID NO: 33 - protein, 201 aa, Homo sapiens
  MISC_FEATURE: Proteasome subunit beta type-2
MEYLIGIQGPDYVLVASDRVAASNIVQMKDDHDKMFKMSEKILLLCVGEAGDTVQFAEYIQKNVQLYKMRNGYELSPTAAANFTRRNLADCLRSRTPYHVNLLLAGYDEHEGPALYYMDYLAALAKAPFAAHGYGAFLTLSILDRYYTPTISRERAVELLRKCLEELQKRFILNLPTFSVRIIDKNGIHDLDNISFPKQGS

SEQ ID NO: 34 - protein, 602 aa, Homo sapiens
  MISC_FEATURE: Cytoskeleton-associated protein 4
MPSAKQRGSKGGHGAASPSEKGAHPSGGADDVAKKPPPAPQQPPPPPAPHPQQHPQQHPQNQAHGKGGHRGGGGGGGKSSSSSSASAAAAAAAASSSASCSRRLGRALNFLFYLALVAAAAFSGWCVHHVLEEVQQVRRSHQDFSRQREELGQGLQGVEQKVQSLQATFGTFESILRSSQHKQDLTEKAVKQGESEVSRISEVLQKLQNEILKDLSDGIHVVKDARERDFTSLENTVEERLTELTKSINDNIAIFTEVQKRSQKEINDMKAKVASLEESEGNKQDLKALKEAVKEIQTSAKSREWDMEALRSTLQTMESDIYTEVRELVSLKQEQQAFKEAADTERLALQALTEKLLRSEESVSRLPEEIRRLEEELRQLKSDSHGPKEDGGFRHSEAFEALQQKSQGLDSRLQHVEDGVLSMQVASARQTESLESLLSKSQEHEQRLAALQGRLEGLGSSEADQDGLASTVRSLGETQLVLYGDVEELKRSVGELPSTVESLQKVQEQVHTLLSQDQAQAARLPPQDFLDRLSSLDNLKASVSQVEADLKMLRTAVDSLVAYSVKIETNENNLESAKGLLDDLRNDLDRLFVKVEKIHEKV

SEQ ID NO: 35 - protein, 367 aa, Homo sapiens
  MISC_FEATURE: Uroporphyrinogen decarboxylase
MEANGLGPQGFPELKNDTFLRAAWGEETDYTPVWCMRQAGRYLPEFRETRAAQDFFSTCRSPEACCELTLQPLRRFPLDAAIIFSDILVVPQALGMEVTMVPGKGPSFPEPLREEQDLERLRDPEVVASELGYVFQAITLTRQRLAGRVPLIGFAGAPWTLMTYMVEGGGSSTMAQAKRWLYQRPQASHQLLRILTDALVPYLVGQVVAGAQALQLFESHAGHLGPQLFNKFALPYIRDVAKQVKARLREAGLAPVPMIIFAKDGHFALEELAQAGYEVVGLDWTVAPKKARECVGKTVTLQGNLDPCALYASEEEIGQLVKQMLDDFGPHRYIANLGHGLYPDMDPEHVGAFVDAVHKHSRLLRQN

SEQ ID NO: 36 - protein, 180 aa, Homo sapiens
  MISC_FEATURE: Adenine phosphoribosyltransferase
MADSELQLVEQRIRSFPDFPTPGVVFRDISPVLKDPASFRAAIGLLARHLKATHGGRIDYIAGLDSRGFLFGPSLAQELGLGCVLIRKRGKLPGPTLWASYSLEYGKAELEIQKDALEPGQRVVVVDDLLATGGTMNAACELLGRLQAEVLECVSLVELTSLKGREKLAPVPFFSLLQYE

SEQ ID NO: 37 - protein, 140 aa, Homo sapiens
  MISC_FEATURE: Profilin-1
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 38 - protein, 627 aa, Homo sapiens [sequence omitted]

SEQ ID NO: 39 - protein, 217 aa, Homo sapiens [sequence omitted]

SEQ ID NO: 40 - protein, 589 aa, Homo sapiens
  MISC_FEATURE: Heterogeneous nuclear ribonucleoprotein L
MSRRLLPRAEKRRRRLEQRQQPDEQRRRSGAMVKMAAAGGGGGGGRYYGGGSEGGRAPKRLKTDNAGDQHGGGGGGGGGAGAAGGGGGGENYDDPHKTPASPVVHIRGLIDGVVEADLVEALQEFGPISYVVVMPKKRQALVEFEDVLGACNAVNYAADNQIYIAGHPAFVNYSTSQKISRPGDSDDSRSVNSVLLFTILNPIYSITTDVLYTICNPCGPVQRIVIFRKNGVQAMVEFDSVQSAQRAKASLNGADIYSGCCTLKIEYAKPTRLNVFKNDQDTWDYTNPNLSGQGDPGSNPNKRQRQPPLLGDHPAEYGGPHGGYHSHYHDEGYGPPPPHYEGRRMGPPVGGHRRGPSRYGPQYGHPPPPPPPPEYGPHADSPVLMVYGLDQSKMNCDRVFNVFCLYGNVEKVKFMKSKPGAAMVEMADGYAVDRAITHLNNNFMFGQKLNVCVSKQPAIMPGQSYGLEDGSCSYKDFSESRNNRFSTPEQAAKNRIQHPSNVLHFFNAPLEVTEENFFEICDELGVKRPSSVKVFSGKSERSSSGLLEWESKSDALETLGFLNHYQMKNPNGPYPYTLKLCFSTAQHAS

SEQ ID NO: 41 - protein, 331 aa, Homo sapiens
  MISC_FEATURE: Reticulocalbin-1 precursor
MARGGRGRRLGLALGLLLALVLAPRVLRAKPTVRKERVVRPDSELGERPPEDNQSFQYDHEAFLGKEDSKTFDQLTPDESKERLGKIVDRIDNDGDGFVTTEELKTWIKRVQKRYIFDNVAKVWKDYDRDKDDKISWEEYKQATYGYYLGNPAEFHDSSDHHTFKKMLPRDERRFKAADLNGDLTATREEFTAFLHPEEFEHMKEIVVLETLEDIDKNGDGFVDQDEYIADMFSHEENGPEPDWVLSEREQFNEFRDLNKDGKLDKDEIRHWILPQDYDHAQAEARHLVYESDKNKDEKLTKEEILENWNMFVGSQATNYGEDLTKNHDEL

SEQ ID NO: 42 - protein, 376 aa, Homo sapiens
  MISC_FEATURE: Serpin B9
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 43 - protein, 366 aa, Homo sapiens
  MISC_FEATURE: Isocitrate dehydrogenase [NAD] subunit alpha mitochondrial precursor
MAGPAWISKVSRLLGAFHNPKQVTRGFTGGVQTVTLIPGDGIGPEISAAVMKIFDAAKAPIQWEERNVTAIQGPGGKWMIPSEAKESMDKNKMGLKGPLKTPIAAGHPSMNLLLRKTFDLYANVRPCVSIEGYKTPYTDVNIVTIRENTEGEYSGIEHVIVDGVVQSIKLITEGASKRIAEFAFEYARNNHRSNVTAVHKANIMRMSDGLFLQKCREVAESCKDIKFNEMYLDTVCLNMVQDPSQFDVLVMPNLYGDILSDLCAGLIGGLGVTPSGNIGANGVAIFESVHGTAPDIAGKDMANPTALLLSAVMMLRHMGLFDHAARIEAACFATIKDGKSLTKDLGGNAKCSDFTEEICRRVKDLD

SEQ ID NO: 44 - protein, 693 aa, Homo sapiens
  MISC_FEATURE: GMP synthase [glutamine hydrolyzing]
MALCNGDSKLENAGGDLKDGHHHYEGAVVILDAGAQYGKVIDRRVRELFVQSEIFPLETPAFAIKEQGFRAIIISGGPNSVYAEDAPWFDPAIFTIGKPVLGICYGMQMMNKVFGGTVHKKSVREDGVFNISVDNTCSLFRGLQKEEVVLLTHGDSVDKVADGFKVVARSGNIVAGIANESKKLYGAQFHPEVGLTENGKVILKNFLYDIAGCSGTFTVQNRELECIREIKERVGTSKVLVLLSGGVDSTVCTALLNRALNQEQVIAVHIDNGFMRKRESQSVEEALKKLGIQVKVINAAHSFYNGTTTLPISDEDRTPRKRISKTLNMTTSPEEKRKIIGDTFVKIANEVIGEMNLKPEEVFLAQGTLRPDLIESASLVASGKAELIKTHHNDTELIRKLREEGKVIEPLKDFHKDEVRILGRELGLPEELVSRHPFPGPGLAIRVICAEEPYICKDFPETNNILKIVADFSASVKKPHTLLQRVKACTTEEDQEKLMQITSLHSLNAFLLPIKTVGVQGDCRSYSYVCGISSKDEPDWESLIFLARLIPRMCHNVNRVVYIFGPPVKEPPTDVTPTFLTTGVLSTLRQADFEAHNILRESGYAGKISQMPVILTPLHFDRDPLQKQPSCQRSVVIRTFITSDFMTGIPATPGNEIPVEVVLKMVTEIKKIPGISRIMYDLTSKPPGTTEWE

SEQ ID NO: 45 - protein, 531 aa, Homo sapiens
  MISC_FEATURE: T-complex protein 1 subunit zeta
MAAVKTLNPKAEVARAQAALAVNISAARGLQDVLRTNLGPKGTMKMLVSGAGDIKLTKDGNVLLHEMQIQHPTASLIAKVATAQDDITGDGTTSNVLIIGELLKQADLYISEGLHPRIITEGFEAAKEKALQFLEEVKVSREMDRETLIDVARTSLRTKVHAELADVLTEAVVDSILAIKKQDEPIDLFMIEIMEMKHKSETDTSLIRGLVLDHGARHPDMKKRVEDAYILTCNVSLEYEKTEVNSGFFYKSAEEREKLVKAERKFIEDRVKKIIELKRKVCGDSDKGFVVINQKGIDPFSLDALSKEGIVALRRAKRRNMERLTLACGGVALNSFDDLSPDCLGHAGLVYEYTLGEEKFTFIEKCNNPRSVTLLIKGPNKHTLTQIKDAVRDGLRAVKNAIDDGCVVPGAGAVEVAMAEALIKHKPSVKGRAQLGVQAFADALLIIPKVLAQNSGFDLQETLVKIQAEHSESGQLVGVDLNTGEPMVAAEVGVWDNYCVKKQLLHSCTVIATNILLVDEIMRAGMSSLKG

SEQ ID NO: 46 - protein, 166 aa, Homo sapiens
  MISC_FEATURE: Cofilin-1
MASGVAVSDGVIKVFNDMKVRKSSTPEEVKKRKKAVLFCLSEDKKNIILEEGKEILVGDVGQTVDDPYATFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 47 - protein, 780 aa, Homo sapiens
  MISC_FEATURE: Aconitate hydratase mitochondrial precursor
MAPYSLLVTRLQKALGVRQYHVASVLCQRAKVAMSHFEPNEYIHYDLLEKNINIVRKRLNRPLTLSEKIVYGHLDDPASQEIERGKSYLRLRPDRVAMQDATAQMAMLQFISSGLSKVAVPSTIHCDHLIEAQVGGEKDLRRAKDINQEVYNFLATAGAKYGVGFWKPGSGIIHQIILENYAYPGVLLIGTDSHTPNGGGLGGICIGVGGADAVDVMAGIPWELKCPKVIGVKLTGSLSGWSSPKDVILKVAGILTVKGGTGAIVEYHGPGVDSISCTGMATICNMGAEIGATTSVFPYNHRMKKYLSKTGREDIANLADEFKDHLVPDPGCHYDQLIEINLSELKPHINGPFTPDLAHPVAEVGKVAEKEGWPLDIRVGLIGSCTNSSYEDMGRSAAVAKQALAHGLKCKSQFTITPGSEQIRATIERDGYAQILRDLGGIVLANACGPCIGQWDRKDIKKGEKNTIVTSYNRNFTGRNDANPETHAFVTSPEIVTALAIAGTLKFNPETDYLTGTDGKKFRLEAPDADELPKGEFDPGQDTYQHPPKDSSGQHVDVSPTSQRLQLLEPFDKWDGKDLEDLQILIKVKGKCTTDHISAAGPWLKFRGHLDNISNNLLIGAINIENGKANSVRNAVTQEFGPVPDTARYYKKHGIRWVVIGDENYGEGSSREHAALEPRHLGGRAIITKSFARIHETNLKKQGLLPLTFADPADYNKIHPVDKLTIQGLKDFTPGKPLKCIIKHPNGTQETILLNHTFNETQIEWFRAGSALNRMKELQQ

SEQ ID NO: 48 - protein, 758 aa, Homo sapiens
  MISC_FEATURE: Mitochondrial inner membrane protein
MLRACQLSGVTAAAQSCLCGKFVLRPLRPCRRYSTSGSSGLTTGKIAGAGLLFVGGGIGGTILYAKWDSHFRESVEKTIPYSDKLFEMVLGPAAYNVPLPKKSIQSGPLKISSVSEVMKESKQPASQLQKQKGDTPASATAPTEAAQIISAAGDTLSVPAPAVQPEESLKTDHPEIGEGKPTPALSEEASSSSIRERPPEEVAARLAQQEKQEQVKIESLAKSLEDALRQTASVTLQAIAAQNAAVQAVNAHSNILKAAMDNSEIAGEKKSAQWRTVEGALKERRKAVDEAADALLKAKEELEKMKSVIENAKKKEVAGAKPHITAAEGKLHNMIVDLDNVVKKVQAAQSEAKVVSQYHELVVQARDDFKRELDSITPEVLPGWKGMSVSDLADKLSTDDLNSLIAHAHRRIDQLNRELAEQKATEKQHITLALEKQKLEEKRAFDSAVAKALEHHRSEIQAEQDRKIEEVRDAMENEMRTQLRRQAAAHTDHLRDVLRVQEQELKSEFEQNLSEKLSEQELQFRRLSQEQVDNFTLDINTAYARLRGIEQAVQSHAVAEEEARKAHQLWLSVEALKYSMKTSSAETPTIPLGSAVEAIKANCSDNEFTQALTAAIPPESLTRGVYSEETLRARFYAVQKLARRVAMIDETRNSLYQYFLSYLQSLLLFPPQQLKPPPELCPEDINTFKLLSYASYCIEHGDLELAAKFVNQLKGESRRVAQDWLKEARMTLETKQIVEILTAYASAVGIGTTQVQPE

SEQ ID NO: 49 - protein, 463 aa, Homo sapiens; identical to SEQ ID NO: 8
  MISC_FEATURE: Heterogeneous nuclear ribonucleoprotein K

SEQ ID NO: 50 - protein, 452 aa, Homo sapiens
  MISC_FEATURE: Elongation factor Tu mitochondrial precursor
MAAATLLRATPHFSGLAAGRTFLLQGLLRLLKAPALPLLCRGLAVEAKKTYVRDKPHVNVGTIGHVDHGKTTLTAAITKILAEGGGAKFKKYEEIDNAPEERARGITINAAHVEYSTAARHYAHTDCPGHADYVKNMITGTAPLDGCILVVAANDGPMPQTREHLLLARQIGVEHVVVYVNKADAVQDSEMVELVELEIRELLTEFGYKGEETPVIVGSALCALEGRDPELGLKSVQKLLDAVDTYIPVPARDLEKPFLLPVEAVYSVPGRGTVVTGTLERGILKKGDECELLGHSKNIRTVVTGIEMFHKSLERAEAGDNLGALVRGLKREDLRRGLVMVKPGSIKPHQKVEAQVYILSKEEGGRHKPFVSHFMPVMFSLTWDMACRIILPPEKELAMPGEDLKFNLILRQPMILEKGQRFTLRDGNRTIGTGLVTNTLAMTEEEKNIKWG

SEQ ID NO: 51 - protein, 325 aa, Homo sapiens
  MISC_FEATURE: Alcohol dehydrogenase [NADP+]
MAASCVLLHTGQKMPLIGLGTWKSEPGQVKAAVKYALSVGYRHIDCAAIYGNEPEIGEALKEDVGPGKAVPREELFVTSKLWNTKHHPEDVEPALRKTLADLQLEYLDLYLMHWPYAFERGDNPFPKNADGTICYDSTHYKETWKALEALVAKGLVQALGLSNFNSRQIDDILSVASVRPAVLQVECHPYLAQNELIAHCQARGLEVTAYSPLGSSDRAWRDPDEPVLLEEPVVLALAEKYGRSPAQILLRWQVQRKVICIPKSITPSRILQNIKVFDFTFSPEEMKQLNALNKNWRYIVPMLTVDGKRVPRDAGHPLYPFNDPY

SEQ ID NO: 52 - protein, 359 aa, Homo sapiens
  MISC_FEATURE: Sialic acid synthase
MPLELELCPGRWVGGQHPCFIIAEIGQNHQGDLDVAKRMIRMAKECGADCAKFQKSELEFKFNRKALERPYTSKHSWGKTYGEHKRHLEFSHDQYRELQRYAEEVGIFFTASGMDEMAVEFLHELNVPFFKVGSGDTNNFPYLEKTAKKGRPMVISSGMQSMDTMKQVYQIVKPLNPNFCFLQCTSAYPLQPEDVNLRVISEYQKLFPDIPIGYSGHETGIAISVAAVALGAKVLERHITLDKTWKGSDHSASLEPGELAELVRSVRLVERALGSPTKQLLPCEMACNEKLGKSVVAKVKIPEGTILTMDMLTVKVGEPKGYPPEDIFNLVGKKVLVTVEEDDTIMEELVDNHGKKIKS

SEQ ID NO: 53 - protein, 282 aa, Homo sapiens
  MISC_FEATURE: S-formylglutathione hydrolase
MALKQISSNKCFGGLQKVFEHDSVELNCKMKFAVYLPPKAETGKCPALYWLSGLTCTEQNFISKSGYHQSASEHGLVVIAPDTSPRGCNIKGEDESWDFGTGAGFYVDATEDPWKTNYRMYSYVTEELPQLINANFPVDPQRMSIFGHSMGGHGALICALKNPGKYKSVSAFAPICNPVLCPWGKKAFSGYLGTDQSKWKAYDATHLVKSYPGSQLDILIDQGKDDQFLLDGQLLPDNFIAACTEKKIPVVFRLQEGYDHSYYFIATFITDHIRHHAKYLNA

SEQ ID NO: 54 - protein, 317 aa, Homo sapiens [sequence omitted]

SEQ ID NO: 55 - protein, 289 aa, Homo sapiens
  MISC_FEATURE: Purine nucleoside phosphorylase
MENGYTYEDYKNTAEWLLSHTKHRPQVAIICGSGLGGLTDKLTQAQIFDYGEIPNFPRSTVPGHAGRLVFGFLNGRACVMMQGRFHMYEGYPLWKVTFPVRVFHLLGVDTLVVTNAAGGLNPKFEVGDIMLIRDHINLPGFSGQNPLRGPNDERFGDRFPAMSDAYDRTMRQRALSTWKQMGEQRELQEGTYVMVAGPSFETVAECRVLQKLGADAVGMSTVPEVIVARHCGLRVFGFSLITNKVIMDYESLEKANHEEVLAAGKQAAQKLEQFVSILMASIPLPDKAS

SEQ ID NO: 56 - protein, 272 aa, Homo sapiens
  MISC_FEATURE: Prohibitin
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIFTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ

SEQ ID NO: 57 - protein, 282 aa, Homo sapiens
  MISC_FEATURE: Complement component 1, Q subcomponent binding protein mitochondrial precursor
MLPLLRCVPRVLGSSVAGLRAAAPASPFRQLLQPAPRLCTRPFGLLSVRAGSERRPGLLRPRGPCACGCGCGSLHTDGDKAFVDFLSDEIKEERKIQKHKTLPKMSGGWELELNGTEAKLVRKVAGEKITVTFNINNSIPPTFDGEEEPSQGQKVEEQEPELTSTPNFVVEVIKNDDGKKALVLDCHYPEDEVGQEDEAESDIFSIREVSFQSTGESEWKDTNYTLNTDSLDWALYDHLMDFLADRGVDNTFADELVELSTALEHQEYITFLEDLKSFVKSQ

SEQ ID NO: 58 - protein, 806 aa, Homo sapiens
  MISC_FEATURE: Endoplasmic reticulum ATPase
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYG

SEQ ID NO: 59 - protein, 152 aa, Homo sapiens
  MISC_FEATURE: Nucleoside diphosphate kinase A
MANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 60 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 61 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 62 - protein, 644 aa, Homo sapiens
MADYSTVPPPSSGSAGGGGGGGGGGGVNDAFKDALQRARQIAAKIGGDAGTSLNSNDYGYGGQKRPLEDGDQPDAKKVAPQNDSFGTQLPPMHQQQSRSVMTEEYKVPDGMVGFIIGRGGEQISRIQQESGCKIQIAPDSGGLPERSCMLTGTPESVQSAKRLLDQIVEKGRPAPGFHHGDGPGNAVQEIMIPASKAGLVIGKGGETIKQLQERAGVKMVMIQDGPQNTGADKPLRITGDPYKVQQAKEMVLELIRDQGGFREVRNEYGSRIGGNEGIDVPIPRFAVGIVIGRNGEMIKKIQNDAGVRIQFKPDDGTTPERIAQITGPPDRCQHAAEIITDLLRSVQAGNPGGPGPGGRGRGRGQGNWNMGPPGGLQEFNFIVPTGKTGLIIGKGGETIKSISQQSGARIELQRNPPPNADPNMKLFTIRGTPQQIDYARQLIEEKIGGPVNPLGPPVPHGPHGVPGPHGPPGPPGPGTPMGPYNPAPYNPGPPGPAPHGPPAPYAPQGWGNAYPHWQQQAPPDPAKAGTDPNSAAWAAYYAHYYQQQAQPPPAAPAGAPTTTQTNGQGDQQNPAPAGQVDYTKAWEEYYKKMGQAVPAPTGAPPGGQPDYSAAWAEYYRQQAAYYAQTSPQGMPQHPPAPQGQ

SEQ ID NO: 63 - protein, 509 aa, Homo sapiens
MQSWSRVYCSLAKRGHFNRISHGLQGLSAVPLRTYADQPIDADVTVIGSGPGGYVAAIKAAQLGFKTVCIEKNETLGGTCLNVGCIPSKALLNNSHYYHMAHGKDFASRGIEMSEVRLNLDKMMEQKSTAVKALTGGIAHLFKQNKVVHVNGYGKITGKNQVTATKADGGTQVIDTKNILIATGSEVTPFPGITIDEDTIVSSTGALSLKKVPEKMVVIGAGVIGVELGSVWQRLGADVTAVEFLGHVGGVGIDMEISKNFQRILQKQGFKFKLNTKVTGATKKSDGKIDVSIEAASGGKAEVITCDVLLVCIGRRPFTKNLGLEELGIELDPRGRIPVNTRFQTKIPNIYAIGDVVAGPMLAHKAEDEGIICVEGMAGGAVHIDYNCVPSVIYTHPEVAWVGKSEEQLKEEGIEYKVGKFPFAANSRAKTNADTDGMVKILGQKSTDRVLGAHILGPGAGEMVNEAALALEYGASCEDIARVCHAHPTLSEAFREANLAASFGKSINF

SEQ ID NO: 64 - protein, 514 aa, Homo sapiens
MADYLISGGTSYVPDDGLTAQQLFNCGDGLTYNDFLILPGYIDFTADQVDLTSALTKKITLKTPLVSSPMDTVTEAGMAIAMALTGGIGFIHHNCTPEFQANEVRKVKKYEQGFITDPVVLSPKDRVRDVFEAKARHGFCGIPITDTGRMGSRLVGIISSRDIDFLKEEEHDCFLEEIMTKREDLVVAPAGITLKEANEILQRSKKGKLPIVNEDDELVAIIARTDLKKNRDYPLASKDAKKQLLCGAAIGTHEDDKYRLDLLAQAGVDVVVLDSSQGNSIFQINMIKYIKDKYPNLQVIGGNVVTAAQAKNLIDAGVDALRVGMGSGSICITQEVLACGRPQATAVYKVSEYARRFGVPVIADGGIQNVGHIAKALALGASTVMMGSLLAATTEAPGEYFFSDGIRLKKYRGMGSLDAMDKHLSSQNRYFSEADKIKVAQGVSGAVQDKGSIHKFVPYLIAGIQHSCQDIGAKSLTQVRAMMYSGELKFEKRTSSAQVEGGVHSLHSYEKRLF

SEQ ID NO: 65 - protein, 510 aa, Homo sapiens
MYRALRLLARSRPLVRAPAAALASAPGLGGAAVPSFWPPNAARMASQNSFRIEYDTFGELKVPNDKYYGAQTVRSTMNFKIGGVTERMPTPVIKAFGILKRAAAEVNQDYGLDPKIANAIMKAADEVAEGKLNDHFPLVVWQTGSGTQTNMNVNEVISNRAIEMLGGELGSKIPVHPNDHVNKSQSSNDTFPTAMHIAAAIEVHEVLLPGLQKLHDALDAKSKEFAQIIKIGRTHTQDAVPLTLGQEFSGYVQQVKYAMTRIKAAMPRIYELAAGGTAVGTGLNTRIGFAEKVAAKVAALTGLPFVTAPNKFEALAAHDALVELSGAMNTTACSLMKIANDIRFLGSGPRSGLGELILPENEPGSSIMPGKVNPTQCEAMTMVAAQVMGNHVAVTVGGSNGHFELNVFKPMMIKNVLHSARLLGDASVSFTENCVVGIQANTERINKLMNESLMLVTALNPHIGYDKAAKIAKTAHKNGSTLKETAIELGYLTAEQFDEWVKPKDMLGPK

SEQ ID NO: 66 - protein, 355 aa, Homo sapiens; identical to SEQ ID NO: 28

SEQ ID NO: 67 - protein, 329 aa, Homo sapiens
MTTQQIDLQGPGPWGFRLVGGKDFEQPLAISRVTPGSKAALANLCIGDVITAIDGENTSNMTHLEAQNRIKGCTDNLTLTVARSEHKVWSPLVTEEGKRHPYKMNLASEPQEVLHIGSAHNRSAMPFTASPASSTTARVITNQYNNPAGLYSSENISNFNNALESKTAASGVEANSRPLDHAQPPSSLVIDKESEVYKMLQEKQELNEPPKQSTSFLVLQEILESEEKGDPNKPSGFRSVKAPVTKVAASIGNAQKLPMCDKCGTGIVGVFVKLRDRHRHPECYVCTDCGTNLKQKGHFFVEDQIYCEKHARERVTPPEGYEVVTVFPK

SEQ ID NO: 68 - protein, 317 aa, Homo sapiens
MPREDRATWKSNYFLKIIQLLDDYPKCFIVGADNVGSKQMQQIRMSLRGKAVVLMGKNTMMRKAIRGHLENNPALEKLLPHIRGNVGFVFTKEDLTEIRDMLLANKVPAAARAGAIAPCEVTVPAQNTGLGPEKTSFFQALGITTKISRGTIEILSDVQLIKTGDKVGASEATLLNMLNISPFSFGLVIQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 69 - protein, 294 aa, Homo sapiens
MATHGQTCARPMCIPPSYADLGKAARDIFNKGFGFGLVKLDVKTKSCSGVEFSTSGSSNTDTGKVTGTLETKYKWCEYGLTFTEKWNTDNTLGTEIAIEDQICQGLKLTFDTTFSPNTGKKSGKIKSSYKRECINLGCDVDFDFAGPAIHGSAVFGYEGWLAGYQMTFDSAKSKLTRNNFAVGYRTGDFQLHTNVNDGTEFGGSIYQKVCEDLDTSVNLAWTSGTNCTRFGIAAKYQLDPTASISAKVNNSSLIGVGYTQTLRPGVKLTLSALVDGKSINAGGHKVGLALELEA

SEQ ID NO: 70 - protein, 189 aa, Homo sapiens
MASKRALVILAKGAEEMETVIPVDVMRRAGIKVTVAGLAGKDPVQCSRDVVICPDASLEDAKKEGPYDVVVLPGGNLGAQNLSESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 71 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 72 - protein, 256 aa, Homo sapiens
MAAAVGRLLRASVARHVSAIPWGISATAALRPAACGRTSLTNLLCSGSSQAKLFSTSSSCHAPAVTQHAPYFKGTAVVNGEFKDLSLDDFKGKYLVLFFYPLDFTFVCPTEIVAFSDKANEFHDVNCEVVAVSVDSHFSHLAWINTPRKNGGLGHMNIALLSDLTKQISRDYGVLLEGSGLALRGLFIIDPNGVIKHLSVNDLPVGRSVEETLRLVKAFQYVETHGEVCPANWTPDSPTIKPSPAASKEYFQKVNQ

SEQ ID NO: 73 - protein, 358 aa, Homo sapiens
MAPQNLSTFCLLLLYLIGAVIAGRDFYKILGVPRSASIKDIKKAYRKLALQLHPDRNPDDPQAQEKFQDLGAAYEVLSDSEKRKQYDTYGEEGLKDGHQSSHGDIFSHFFGDFGFMFGGTPRQQDRNIPRGSDIIVDLEVTLEEVYAGNFVEVVRNKPVARQAPGKRKCNCRQEMRTTQLGPGRFQMTQEVVCDECPNVKLVNEERTLEVEIEPGVRDGMEYPFIGEGEPHVDGEPGDLRFRIKVVKHPIFERRGDDLYTNVTISLVESLVGFEMDITHLDGHKVHISRDKITRPGAKLWKKGEGLPNFDNNNIKGSLIITFDVDFPKEQLTEEAREGIKQLLKQGSVQKVYNGLQGY

SEQ ID NO: 74 - protein, 335 aa, Homo sapiens
MAAGAAEAAVAAVEEVGSAGQFEELLRLKAKSLLVVHFWAPWAPQCAQMNEVMAELAKELPQVSFVKLEAEGVPEVSEKYEISSVPTFLFFKNSQKIDRLDGAHAPELTKKVQRHASSGSFLPSANEHLKEDLNLRLKKLTHAAPCMLFMKGTPQEPRCGFSKQMVEILHKHNIQFSSFDIFSDEEVRQGLKAYSSWPTYPQLYVSGELIGGLDIIKELEASEELDTICPKAPKLEERLKVLTNKASVMLFMKGNKQEAKCGFSKQILEILNSTGVEYETFDILEDEEVRQGLKAYSNWPTYPQLYVKGELVGGLDIVKELKENGELLPILRGEN

SEQ ID NO: 75 - protein, 201 aa, Homo sapiens
MTEKAPEPHVEEDDDDELDSKLNYKPPPQKSLKELQEMDKDDESLIKYKKTLLGDGPVVTDPKAPNVVVTRLTLVCESAPGPITMDLTGDLEALKKETIVLKEGSEYRVKIHFKVNRDIVSGLKYVQHTYRTGVKVDKATFMVGSYGPRPEEYEFLTPVEEAPKGMLARGTYHNKSFFTDDDKQDHLSWEWNLSIKKEWTE

SEQ ID NO: 76 - protein, 210 aa, Homo sapiens
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 77 - protein, 198 aa, Homo sapiens
MASGNARIGKPAPDFKATAVVDGAFKEVKLSDYKGKYVVLFFYPLDFTFVCPTEIIAFSNRAEDFRKLGCEVLGVSVDSQFTHLAWINTPRKEGGLGPLNIPLLADVTRRLSEDYGVLKTDEGIAYRGLFIIDGKGVLRQITVNDLPVGRSVDEALRLVQAFQYTDEHGEVCPAGWKPGSDTIKPNVDDSKEYFSKHN

SEQ ID NO: 78 - protein, 343 aa, Homo sapiens
MPEETQTQDQPMEEEEVETFAFQAEIAQLMSLIINTFYSNKEIFLRELISNSSDALDKIWYESLTDPSKLDSGKELHINLIPNKQDQTLTIVDTGIGMTKADLINNLGTIAKSGTKAFMEALQAGADISMIGQFGVSFYSAYLVAEKVTVITKHNDDEQYAWESSAGGSFTVRTDTGERMGRGTKVILHLKEDQTEYLEEQRIKEIVKKHSQLIGYPITLFVEKECDKEVSDDETEEKEDKEEEKEKEEKESKDKPEIEDVGSDEEEEKKDGDKKKKKTKEKYIDQEELNKTKPIWTRNPDDITNEEYGEFCKNLTNDWEDHLAVKHFSVEGQLEFRALLFVP

SEQ ID NO: 79 - protein, 1224 aa, Homo sapiens
MLTKFETKSARVKGLSFHPKRPWILTSLHNGVIQLWDYRMCTLIDKFDEHDGPVRGIDFHKQQPLFVSGGDDYKIKVWNYKLRRCLFTLLGHLDYIRTTFFHHEYPWILSASDDQTIRVWNWQSRTCVCVLTGHNHYVMCAQFHPTEDLVVSASLDQTVRVWDISGLRKKNLSPGAVESDVRGITGVDLFGTTDAVVKHVLEGHDRGVNWAAFHPTMPLIVSGADDRQVKIWRMNESKAWEVDTCRGHYNNVSCAVFHPRQELILSNSEDKSIRVWDMSKRTGVQTFRRDHDRFWVLAAHPNLNLFAAGHDGGMIVFKLERERPAYAVHGNMLHYVKDRFLRQLDFNSSKDVAVMQLRSGSKFPVFNMSYNPAENAVLLCTRASNLENSTYDLYTIPKDADSQNPDAPEGKRSSGLTAVWVARNRFAVLDRMHSLLIKNLKNEITKKVQVPNCDEIFYAGTGNLLLRDADSITLFDVQQKRTLASVKISKVKYVIWSADMSHVALLAKHAIVICNRKLDALCNIHENIRVKSGAWDESGVFIYTTSNHIKYAVTTGDHGIIRTLDLPIYVTRVKGNNVYCLDRECRPRVLTIDPTEFKFKLALINRKYDEVLHMVRNAKLVGQSIIAYLQKKGYPEVALHFVKDEKTRFSLALECGNIEIALEAAKALDDKNCWEKLGEVALLQGNHQIVEMCYQRTKNFDKLSFLYLITGNLEKLRKMMKIAEIRKDMSGHYQNALYLGDVSERVRILKNCGQKSLAYLTAATHGLDEEAESLKETFDPEKETIPDIDPNAKLLQPPAPIMPLDTNWPLLTVSKGFFEGTIASKGKGGALAADIDIDTVGTEGWGEDAELQLDEDGFVEATEGLGDDALGKGQEEGGGWDVEEDLELPPELDISPGAAGGAEDGFFVPPTKGTSPTQIWCNNSQLPVDHILAGSFETAMRLLHDQVGVIQFGPYKQLFLQTYARGRTTYQALPCLPSMYGYPNRNWKDAGLKNGVPAVGLKLNDLIQRLQLCYQLTTVGKFEEAVEKFRSILLSVPLLVVDNKQEIAEAQQLITICREYIVGLSVETERKKLPKETLEQQKRICEMAAYFTHSNLQPVHMILVLRTALNLFFKLKNFKTAATFARRLLELGPKPEVAQQTRKILSACEKNPTDAYQLNYDMHNPFDICAASYRPIYRGKPVEKCPLSGACYSPEFKGQICRVTTVTEIGKDVIGLRISPLQFR

SEQ ID NO: 80 - protein, 494 aa, Homo sapiens
MFEIKKICCIGAGYVGGPTCSVIAHMCPEIRVTVVDVNESRINAWNSPTLPIYEPGLKEVVESCRGKNLFFSTNIDDAIKEADLVFISVNTPTKTYGMGKGRAADLKYIEACARRIVQNSNGYKIVTEKSTVPVRAAESIRRIFDANTKPNLNLQVLSNPEFLAEGTAIKDLKNPDRVLIGGDETPEGQRAVQALCAVYEHWVPREKILTTNTWSSELSKLAANAFLAQRISSINSISALCEATGADVEEVATAIGMDQRIGNKFLKASVGFGGSCFQKDVLNLVYLCEALNLPEVARYWQQVIDMNDYQRRRFASRIIDSLFNTVTDKKIAILGFAFKKDTGDTRESSSIYISKYLMDEGAHLHIYDPKVPREQIVVDLSHPGVSEDDQVSRLVTISKDPYEACDGAHAVVICTEWDMFKELDYERIHKKMLKPAFIFDGRRVLDGLHNELQTIGFQIETIGKKVSSKRIPYAPSGEIPKFSLQDPPNKKPKV

SEQ ID NO: 81 - protein, 375 aa, Homo sapiens
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 82 - protein, 1075 aa, Homo sapiens
MVVEVDSMPAASSVKKPFGLRSKMGKWCCRCFPCYRESGKSNVGTSGDHDDSAMKTLRSKMGKWCHHCFPCCRGSGKSNVGASGDHDDSAMKTLRNKMGKWCCHCFPCCRGSGKSKVGAWGDYDDSAFMEPRYHVRGEDLDKLHRAAWWGKVPRKDLIVMLRDTDVNKKDKQKRTALHLASANGNSEVVKLLLDRRCQLNVLDNKKRTALIKAVQCQEDECALMLLEHGTDPNIPDEYGNTTLHYAIYNEDKLMAKALLLYGADIESKNKHGLTPLLLGVHEQKQQVVKFLIKKKANLNALDRYGRTALILAVCCGSASIVSLLLEQNIDVSSQDLSGQTAREYAVSSHHHVICQLLSDYKEKQMLKISSENSNPEQELKLTSEEESQRFKGSENSQPEKMSQELEINKDGDREVEEEMKKHESNNVGLLENLTNGVTAGNGDNGLIPQRKSRTPENQQFPDNESEEYHRICELLSDYKEKQMPKYSSENSNPEQDLKLTSEEESQRLKGSENGQPEKRSQEPEINKDGDRELENFMAIEEMKKHGSTHVGFPENLTNGATAGNGDDGLIPPRKSRTPESQQFPDTENEEYHSDEQNDTQKQFCEEQNTGILHDEILIHEEKQIEVVEKMNSELSLSCKKEKDVLHENSTLREEIAMLRLELDTMKHQSQLREKKYLEDIESVKKKNDNLLKALQLNELTMDDDTAVLVIDNGSGMCKAGFAGDDAPRAVFPSIVGRPRQQGMMGGMHQKESYVGKEAQSKRGILTLKYPMEHGIITNWDDMEKIWHHTFYNELRVAPEEHPILLTEAPLNPKANREKMTQIMFETFNTPAMYVAIQAVPSLYTSGRTTGIVMDSGDGVTHTVPIYEGNALPHATLRLDLAGRELPDYLMKILTERGYRFTTMAEREIVRDIKEKLCYVALDFEQEMATAASSSSLEKSYELPDGQVITIGNERFRCPEALFQPCFLGMESCGIHETTFNSIMKSDVDIRKDLYTNTVLSGGTTMYPGMAHRMQKEIAALAPSMMKIRIIAPPKRKYSVWVGGSILASLSTFQQMWISKQEYDESGPSIVHRKCF

SEQ ID NO: 83 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 84 - protein, 858 aa, Homo sapiens
MVNFTVDQIRAIMDKKANIRNMSVIAHVDHGKSTLTDSLVCKAGIIASARAGETRFTDTRKDEQERCITIKSTAISLFYELSENDLNFIKQSKDGAGFLINLIDSPGHVDFSSEVTAALRVTDGALVVVDCVSGVCVQTETVLRQAIAERIKPVLMMNKMDRALLELQLEPEELYQTFQRIVENVNVIISTYGEGESGPMGNIMIDPVLGTVGFGSGLHGWAFTLKQFAEMYVAKFAAKGEGQLGPAERAKKVEDMMKKLWGDRYFDPANGKFSKSATSPEGKKLPRTFCQLILDPIFKVFDAIMNFKKEETAKLIEKLDIKLDSEDKDKEGKPLLKAVMRRWLPAGDALLQMITIHLPSPVTAQKYRCELLYEGPPDDEAAMGIKSCDPKGPLMMYISKMVPTSDKGRFYAFGRVFSGLVSTGLKVRIMGPNYTPGKKEDLYLKPIQRTILMMGRYVEPIEDVPCGNIVGLVGVDQFLVKTGTITTFEHAHNMRVMKFSVSPVVRVAVEAKNPADLPKLVEGLKRLAKSDPMVQCIIEESGEHIIAGAGELHLEICLKDLEEDHACIPIKKSDPVVSYRETVSEESNVLCLSKSPNKHNRLYMKARPFPDGLAEDIDKGEVSARQELKQRARYLAEKYEWDVAEARKIWCFGPDGTGPNILTDITKGVQYLNEIKDSVVAGFQWATKEGALCEENMRGVRFDVHDVTLHADAIHRGGGQIIPTARRCLYASVLTAQPRLMEPIYLVEIQCPEQVVGGIYGVLNRKRGHVFEESQVAGTPMFVVKAYLPVNESFGFTADLRSNTGGQAFPQCVFDHWQILPGDPFDNSSRPSQVVAETRKRKGLKEGIPALDNFLDKL

SEQ ID NO: 85 - protein, 397 aa, Homo sapiens
MVKETTYYDVLGVKPNATQEELKKAYRKLALKYHPDKNPNEGEKFKQISQAYEVLSDAKKRELYDKGGEQAIKEGGAGGGFGSPMDIFDMFFGGGGRMQRERRGKNVVHQLSVTLEDLYNGATRKLALQKNVICDKCEGRGGKKGAVECCPNCRGTGMQIRIHQIGPGMVQQIQSVCMECQGHGERISPKDRCKSCNGRKIVREKKILEVHIDKGMKDGQKITFHGEGDQEPGLEPGDIIIVLDQKDHAVFTRRGEDLFMCMDIQLVEALCGFQKPISTLDNRTIVITSHPGQIVKHGDIKCVLNEGMPIYRRPYEKGRLIIEFKVNFPENGFLSPDKLSLLEKLLPERKEVEETDEMDQVELVDFDPNQERRRHYNGEAYEDDEHHPRGGVQCQTS

SEQ ID NO: 86 - protein, 375 aa, Homo sapiens
MEEEIAALVIDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 87 - protein, 406 aa, Homo sapiens
MALDGPEQMELEEGKAGSGLRQYYLSKIEELQLIVNDKSQNLRRLQAQRNELNAKVRLLREELQLLQEQGSYVGEVVRAMDKKKVLVKVHPEGKFVVDVDKNIDINDVTPNCRVALRNDSYTLHKILPNKVDPLVSLMMVEKVPDSTYEMIGGLDKQIKEIKEVIELPVKHPELFEALGIAQPKGVLLYGPPGTGKTLLARAVAHHTDCTFIRVSGSELVQKFIGEGARMVRELFVMAREHAPSIIFMDEIDSIGSSRLEGGSGGDSEVQRTMLELLNQLDGFEATKNIKVIMATNRIDILDSALLRPGRIDRKIEFPPPNEEARLDILKIHSRKMNLTRGINLRKIAELMPGASGAEVKGVCTEAGMYALRERRVHVTQEDFEMAVAKVMQKDSEKNMSIKKLWK

SEQ ID NO: 88 - protein, 563 aa, Homo sapiens
MGLQACLLGLFALILSGKCSYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP